(19) United States
(12) Patent Application Publication
(10) Pub. No.: US 2025/0231184 A1
(43) Pub. Date: Jul. 17, 2025

(54) NITRILOTRIACETIC ACID LINKERS, SOLID PHASE SYNTHESIS OF NITRILOTRIACETIC ACID LINKERS AND APPLICATIONS THEREOF

(71) Applicant: Nicoya Lifesciences Inc., Kitchener (CA)

(72) Inventor: Hojjat S. JAMALI, Kitchener (ON)

(21) Appl. No.: 18/726,743

(22) PCT Filed: Oct. 11, 2023
(86) PCT No.: PCT/CA2023/051346

§ 371 Date: Jul. 3, 2024

Related U.S. Application Data
(60) Provisional application No. 63/379,207, filed on Oct. 12, 2022.

(51) Int. Cl. G01N 33/543; C07C 237/52; G01N 33/68

(52) U.S. Cl. CPC G01N 33/54373; C07C 237/52; G01N 33/6869; G01N 2333/5412; G01N 2333/7155

(57) ABSTRACT

Described are devices, compounds and methods for detecting an analyte in a sample. More particularly this disclosure provides SPR and LSPR sensors modified by NTA linkers for binding a ligand and/or analyte. The NTA linkers of the disclosure typically include a head (or “anchoring site”) for coupling to a surface of the SPR or LSPR sensor, a spacer, and one or more attachment sites on a distal end or ends of the spacer which couple to a ligand. The head may include a thiol for coupling the linker to a surface of the SPR or LSPR sensor. The spacer may be a carbon chain. PAG or PEG chain, or matrix material. The one or more attachment sites may be a nitrilotriacetic acid (NTA) moiety capable of chelating a transition metal ion and binding the ligand.

### CROSS-REFERENCE

This application claims the benefit of U.S. Provisional Application No. 63/379,207, filed Oct. 12, 2022, which is hereby incorporated by reference in its entirety herein.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### FIELD

The subject matter relates generally to the solid phase synthesis and use of linkers, or nitrilotriacetic acid linkers, for biosensing application using surface plasmon resonance.

### BACKGROUND

Surface plasmon resonance (SPR) and localized surface plasmon resonance (LSPR) spectroscopy are used for a variety of analytical methods such as quantitative, kinetic and thermodynamic studies. SPR and LSPR provide a label-free method of determining molecular binding events real-time for analytes such as DNA, proteins and polymers. Current SPR and LSPR applications use a self-assembled monolayer (SAM) composed of a thiol containing group attached to the gold surface, whether a thin gold film or a gold nanoparticle, with alkane groups extending away from the gold surface and a single carboxylic acid group at the end of the chain.

Detection of protein-protein or protein-ligand interactions using SPR and LSPR are possible through immobilizing a protein analyte on the chip surface using high affinity interactions such as antibody capture, streptavidin/biotin, or covalent coupling using amino groups to directly observe the binding of a ligand to the immobilized component. Once protocols are established for immobilizing a particular analyte on a sensor surface, determination of binding affinities and screening for interaction partners can be performed in an automated manner. However, in our experience, identifying an optimal way to immobilize protein analytes resulting in a stable surface with high binding capacity can be tedious.

Since immobilized metal ion affinity chromatography (IMAC) was first reported, several modifications have been developed. Among them, Ni²⁺ immobilized by chelation with nitrilotriacetic acid (NTA) bound to a solid support has become the most common method for the purification of proteins carrying histidine (His) tag.

Different types of NTA-based sensors are reported including NTA-modified glass-type surfaces, NTA-functionalized dextran hydrogels and NTA-terminated (alkyl) thiol SAMs as well as solid-supported lipid bilayers doped with NTA-lipids. The availability of commercial nitrilotriacetic acid (NTA) sensor chips allows repeated immobilization, stripping, and regeneration of His6-tagged proteins; however, this method of immobilization has multiple deficiencies that prevent its wide-spread use. Firstly, while the NTA/His6 interaction is robust, slow and continuous dissociation of immobilized components is common. The decaying surface can be corrected post-hoc using computational methods; however, this solution is less than ideal. The second commonly encountered problem with NTA sensor chips is the idiosyncratic drift of flow cells. If equivalent drifts occur across all flow cells, double referencing can be used to correct for systematic deviations across all flow cells; however, we have frequently encountered deviations that only occur on individual flow cells which cannot be corrected for using this method.

### SUMMARY

In some aspects, the present disclosure relates to a nitrilotriacetic acid linker having the formula:

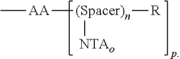

In some embodiments, the AA group includes a linear or branched natural or synthetic amino acid group including one or more amino acids. In some embodiments, the optional spacer includes a linear or branched carbon chain, including one or more amino acids, a polymeric moiety, or any combination thereof. In some embodiments, in the absence of the spacer, the AA group is coupled to the one or more nitrilotriacetic acid (NTA) groups. In some embodiments, the nitrilotriacetic acid (NTA) group includes one or more nitrilotriacetic acid functional groups. In some embodiments, wherein R is H, a short chain alkyl, or an NTA group. In some embodiments, “n” is the number of spacers in the linker. In some embodiments, “n” includes from 0 to 50 spacers. In some embodiments, “o” is the number of NTA groups in the linker. In some embodiments, “o” includes from 1 to 10 NTA groups. In some embodiments, “p” is the number of

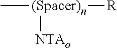

moieties. In some embodiments, “p” includes from 1 to 10 moieties.

In some aspects, the nitrilotriacetic acid linker includes an AA group. In some embodiments, the AA group includes from one to 10 amino acids. In some embodiments, AA group includes a cysteine or a methionine amino acid.

In some aspects, the nitrilotriacetic acid linker includes a spacer. In some embodiments, the spacer includes a carbon chain. In some embodiments, the spacer includes a PAG group having one or more PAG units. In some embodiments, the spacer includes a PEG group having one or more PEG units.

In some embodiments, the one or more NTA groups are configured to couple to a ligand and/or an analyte.

In some aspects, the present disclosure relates to a surface plasmon resonance (SPR) or localized surface plasmon resonance (LSPR) sensor, the sensor including: (a) a substrate, wherein the substrate includes a solid support coated with a metal layer; and (b) a nitrilotriacetic acid (NTA) linker. In some embodiments, the AA group of the NTA linker is coupled to the metal layer. In some embodiments, the one or more nitrilotriacetic acid functional groups of the linker are capable of binding to one or more ligands and/or analytes.

In some embodiments, the sensor includes a substrate and a coating layer. In some embodiments, the AA group is covalently coupled to the coating layer. In some embodiments, the substrate is selected from a group consisting of silicon substrates, glass substrates, polystyrene substrates, agarose substrates. In some embodiments, the coating layer includes a first inner coating and a second outer coating. In some embodiments, the first inner coating includes a polyelectrolyte or a poly(allylamine hydrochloride). In some embodiments, the second outer coating includes a metal coating, a gold film, a metal nanoparticle coating, or a gold nanoparticle coating.

In some aspects, the present disclosure relates to a method for detection of an analyte in a fluid using a surface plasmon resonance (SPR or LSPR) sensor, the method including the steps of: providing an SPR or LSPR sensor including a surface, the surface including a solid support coated with a metal layer and having a nitrilotriacetic acid linker attached to the metal layer; contacting a fluid comprising an analyte with the SPR or LSPR sensor; and measuring an optical signal to detect a change in the optical signal in response to the contacting to measure the analyte in the fluid. In some embodiments, the nitrilotriacetic acid linker is configured to bind one or more ligands.

In some aspects, the present disclosure relates to a method for making a sensor coupled to the nitrilotriacetic acid linker, the method including the steps of: providing a substrate; coupling a first AA group to the substrate to yield substrate-AA; optionally coupling a spacer to the first AA group to yield substrate-AA-Spacer, and the spacer comprises an amino acid at a first terminus of the spacer comprises an amino acid at a first terminus of the spacer; and reacting the terminus amino acid with a first reagent to produce a nitrilotriacetic acid linker.

In some embodiments, the substrate is selected from a group consisting of silicon substrates, glass substrates, polystyrene substrates, agarose substrates.

In some embodiments, the AA group includes a substrate coupling group and an α-amino group.

In some embodiments, the spacer includes a first coupling group on a first site, a second coupling group on a second site, and optionally a third coupling group on a third site. In some embodiments, the first, second and optionally third coupling groups may be the same or different chemical functional groups.

In some embodiments, the first AA group further includes one or more protecting groups. In some embodiments, the one or more protecting groups one the first AA group includes one or more trityl functional groups and/or one or more fmoc functional groups. In some embodiments, the AA group includes a fmoc protected α-amino group, a trityl protected thiol functional group and a carboxyl group which reacts with a substituent on the substrate to covalently bind to the substrate. In some embodiments, the fmoc protected α-amino group of the AA group is removed using a second reagent to yield a deprotected α-amino group.

In some embodiments, the spacer further includes one or more protecting groups. In some embodiments, the one or more protecting groups on the spacer include one or more fmoc functional groups and one or more tert-butyl functional groups. In some embodiments, the spacer includes a fmoc protected amino group and a carboxyl group which reacts with the deprotected α-amino group on the AA group to covalently bind to the AA group. In some embodiments, the fmoc protected amino group of the spacer is removed using a second reagent to yield a functional group, and wherein the functional group is reacted with a second AA to yield a deprotected amino group.

In some embodiments, the second AA includes one or more protecting groups. In some embodiments, the one or more protecting groups on the second AA include one or more fmoc functional groups and/or one or more tert-butyl functional groups. In some embodiments, the second AA includes a fmoc protected α-amino group, the tert-butyl functional group and a carboxyl group which reacts with the deprotected amino group on the spacer to covalently bind to the spacer. In some embodiments, the fmoc protected α-amino group on the second AA is removed to yield a deprotected α-amino group. In some embodiments, the deprotected α-amino group is reacted with a second reagent to yield an NTA-tBu functional group. In some embodiments, the tert-butyl protecting group on the second AA is removed using the first reagent to yield the nitrilotriacetic acid linker.

In some embodiments, the spacer includes a hydrophilicity modification. In some embodiments, the hydrophilicity modification includes a PAG group having one or more PAG units. In some embodiments, the hydrophilicity modification includes a PEG group having one or more PEG units.

In some embodiments, the nitrilotriacetic acid linker is decoupled from the substrate.

In some embodiments, the first AA group includes fmoc-D-Cys(Trt)-OH or Fmoc-D-Met-OH.

In some embodiments, the first AA group includes a series of amino acids where one amino acid contains a moiety for attaching to the substrate and another amino acid contains a moiety with an α amino group adjacent to a carboxyl group.

In some embodiments, the spacer includes fmoc-11-aminoundecanoic acid.

In some embodiments, the second AA includes Fmoc-Glu-OtBu.

In some embodiments, the first reagent includes iodoacetic acid (IAA) and diisopropylethylamine (DIPEA).

In some embodiments, the second reagent includes 20% piperidine in a solution of dimethylformamide (DMF), trifluoroacetic acid (TFA) and triisopropylsilane (TIS) or trifluoroacetic acid (TFA), dimethyl sulfoxide (DMSO) and anisole.

In some aspects, the present disclosure relates to a sensor for reversibly capturing a plurality of analytes in a sample, the sensor including: a metal nanoparticle configured to produce an optical signal; a matrix disposed over a portion of a surface of the metal nanoparticle; and a plurality of polydentate groups bound to the matrix, wherein each polydentate group of the plurality of polydentate groups is chelated to a metal ion.

In some embodiments, the matrix includes a polysaccharide, a polysaccharide derivative, a polymer, a functionalized polymer, a protein, or a combination thereof.

In some embodiments, the matrix includes dextran or carboxymethylated dextran.

In some embodiments, the metal ion includes a transition metal ion.

In some embodiments, the transition metal ion is selected from the group consisting of nickel, iron, cobalt, copper, zinc, or combinations thereof.

In some embodiments, plurality of polydentate groups includes nitrilotriacetic acid, tris-(2-aminoethyl)amine, or a combination thereof.

In some embodiments, the matrix includes dextran, and the plurality of tridentate groups is nitrilotriacetic acid.

In some embodiments, the matrix includes a matrix thickness from about 1 nm to about 100 nm.

In some embodiments, the sensor includes a density of polydentate groups from about 1% to about 100%. In some embodiments, the sensor includes a density of polydentate groups from about 25% to about 75%.

In some embodiments, the matrix is covalently bound to the metal nanoparticle.

In some aspects, the present disclosure relates to a method of forming a sensor medium, the method including the steps of: thiolating a matrix material to provide a thiolated matrix material; adding at least one polydentate group to the thiolated matrix material to provide a modified matrix material; and disposing the modified matrix material over a portion of a surface of a metal substrate.

In some embodiments, the step of thiolating a matrix material to provide a thiolated matrix material precedes adding at least one polydentate group to the thiolated matrix material to provide a modified matrix material.

In some embodiments, the step of thiolating a matrix material includes combining the matrix material with an amine a derivative thereof. In some embodiments, the amine includes a mercaptoalkylamine.

In some embodiments, each of the at least one polydentate group includes —SR, —OR, —NR2, —COOR, —CO, a heterocycloalkyl, or a combination thereof. In some embodiments, R is independently H, a substituted or unsubstituted C1-C4 alkyl, or a combination thereof. In some embodiments, the at least one polydentate group is nitrilotriacetic acid, tris-(2-aminoethyl)amine, tris(hydroxymethyl)aminomethane, or a combination thereof.

In some embodiments, the metal substrate includes a metal nanoparticle.

In some embodiments, the method further includes chelating the at least one polydentate group with a metal ion. In some embodiments, the metal ion includes a transition metal ion selected from the group including nickel, iron, cobalt, copper, calcium, zinc, or combinations thereof.

In some aspects, the present disclosure relates to a method of determining the presence of an analyte in a sample, the method including the steps of: providing a CMD-NTA modified sensor; contacting the sensor with the sample, the sample comprising the analyte; and binding the analyte to the sensor to generate a signal corresponding to the analyte.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 illustrates an exemplary SPR sensor system according to principles described herein.

FIG. 2 illustrates an exemplary LSPR metal nanoparticle sensor system according to principles described herein.

FIG. 3A-C illustrates exemplary SPR or LSPR tunable linker molecules differing in the density of attachment sites and spacer molecules.

FIG. 4 illustrates exemplary SPR or LSPR tunable linker molecules with different amino acids.

FIG. 5 illustrates a sensogram of nanoparticles coated with an NTA-modified carboxymethyldextrose (CMD-NTA) upon exposure to anti-IL-6 antibodies in the presence and absence of IL-6.

FIG. 6 is a graph showing IL-6 ligand immobilization response for four separate channels (i.e., 4 different CMD-NTA modified sensors).

FIG. 7 is a graph showing IL-6 analyte immobilization response for four separate channels (i.e., 4 different CMD-NTA modified sensors).

### DETAILED DESCRIPTION

In the following detailed description, reference is made to the accompanying figures, which form a part hereof. In the figures, similar symbols typically identify similar components, unless context dictates otherwise. The illustrative embodiments described in the detailed description, figures, and claims are not meant to be limiting. Other embodiments may be utilized, and other changes may be made, without departing from the scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

Although certain embodiments and examples are disclosed below, inventive subject matter extends beyond the specifically disclosed embodiments to other alternative embodiments and/or uses, and to modifications and equivalents thereof. Thus, the scope of the claims appended hereto is not limited by any of the particular embodiments described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments, however, the order of description should not be construed to imply that these operations are order dependent. Additionally, the structures, systems, and/or devices described herein may be embodied as integrated components or as separate components.

For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

### Terms, Definitions, and Abbreviations

Unless otherwise defined, all technical terms used herein have the same meaning as commonly understood by one of ordinary skill in the art to which this disclosure belongs.

As used herein, the singular forms “a.” “an.” and “the” include plural references unless the context clearly dictates otherwise. Any reference to “or” herein is intended to encompass “and/or” unless otherwise stated.

As used herein, the term “about” in some cases refers to an amount that is approximately the stated amount.

As used herein, the term “about” refers to an amount that is near the stated amount by 10%, 5%, or 1%, including increments therein.

As used herein, the term “about” in reference to a percentage refers to an amount that is greater or less the stated percentage by 10%, 5%, or 1%, including increments therein.

As used herein, the phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.
“AAA” is the abbreviation for “attachment amino acid.”
“AG” is the abbreviation for “attachment group.”
“Asp” is the abbreviation for aspartic acid.
“AuNPs” is the abbreviation for “gold nanoparticles.”
“Cys” is the abbreviation for cysteine.
“CMD” is the abbreviation for carboxymethyl dextrose.
“Da” is the abbreviation for diazonium salts.
“DIPEA” is the abbreviation for diisopropylethylamine.
“DMF” is the abbreviation for “dimethylformamide.”
“DMSO” is the abbreviation for “dimethyl sulfoxide.”
“EDC” is the abbreviation for N-(3-Dimethylaminopropyl)-N′-ethylcarbodiimide hydrochloride
“fmoc” is the abbreviation for “9-fluorenylmethyloxycarbonyl chloride.”
“fmoc-Glu-OH” is the abbreviation for “fmoc-L-glutamic acid.”
“Glu” is the abbreviation for glutamic acid.
“Gly” is the abbreviation for glycine.
“HAA” is the abbreviation for “head amino acid.”
“HATU” is the abbreviation for “(1-[Bis(dimethylamino)methylene]-1H-1,2,3-triazolo [4,5-b]pyridinium 3-oxide hexafluorophosphate, or hexafluorophosphate azabenzotriazole tetramethyl uronium).
“HG” is the abbreviation for “head group.”
“HIS” is the abbreviation for histidine.
“HM” is the abbreviation for “hydrophilic moiety.”
“HOBT” is the abbreviation for “hydroxybenzotriazole.”
“IAA” is the abbreviation for iodoacetic acid.
“IMAC” is the abbreviation for metal ion affinity chromatography.
“LM” is the abbreviation for “lipophilic moiety.”
“LSPR” is the abbreviation for “localized surface plasmon resonance.”
“Met” is the abbreviation for methionine.
“NPs” is the abbreviation for “nanoparticles.”
“NPC” is the abbreviation for “NH₂-PEG-COOH” or “amine-PEG-carboxyl.”
“NR” is the abbreviation for “nitrogen containing functional group.”
“NSB” is the abbreviation for “non-specific binding.”
“NTA” is the abbreviation for nitrilotriacetic acid.
“Oac” is the abbreviation for an acetyl functional group.
“PAH” is the abbreviation for the polyelectrolyte poly(allylamine hydrochloride).
“PBS” is the abbreviation for “phosphate-buffered saline.”
“PE” is the abbreviation for polyelectrolyte.
“PEG” is the abbreviation for “polyethylene glycol.”
“Pip” is the abbreviation for piperidine.
“RA” is the abbreviation for “reagent alcohol.”
“SAA” is the abbreviation for “spacer amino acid.”
“SG” is the abbreviation for “spacer group.”
“SPR” is the abbreviation for “surface plasmon resonance.”
“SR” is the abbreviation for “sulfur containing functional group.”
“tBu” is the abbreviation for a tert-Butyl functional group.
“TIS” is the abbreviation for triisopropylsilane.
“TFA” is the abbreviation for trifluoroacetic acid.
“Trt” is the abbreviation for a trityl functional group.
“Amino group” means any primary, secondary, tertiary or quaternary amine functional group.
“Analyte” means the chemical of interest which binds to the ligand and this binding is detected by SPR or LSPR.
“Anchoring site” means the location on the chemical (e.g., peptide) chain which attaches to the (e.g., gold) surface.
“Attachment amino acid” means any natural or custom synthesized amino acid with one or more functional groups which act as an attachment site in the linker.
“Attachment group” means any natural or custom synthesized molecule with one or more functional groups which act as an attachment site in the linker.
“Attachment sites” means the locations on the (e.g., peptide) chain (e.g., carboxylic acid) which couple with the ligand.
“Diazonium salts” are chemical moieties of the form R—N₂⁺.
“Head” means the anchoring site (e.g., thiol) of the linker (e.g., peptide chain) which provides a tunable method of anchoring the SAM to the substrate.
“Head amino acid” means any natural or custom synthesized amino acid used as the anchoring site in the head of the linker.
“Head group” means the molecule that acts as an anchoring site (e.g., thiol) of the linker chain which provides a tunable method of anchoring the SAM to the substrate.
“Ligand” means the chemical moiety which may be coupled to the attachment site and which is used to bind to the analyte. The ligand, may for example, be any binder, such as an antibody, aptamer, polymer, DNA or other capture molecule having affinity for an analyte.
“Linker” means the customizable or tunable molecular chain which may form a self-assembled monolayer (SAM).
“Linker body” means the section in the middle of the chemical (e.g., peptide) chain which may be tunable in length, tunable in steric considerations and tunable in the density of attachment sites.
“Nanoparticle” means a particle of varying sizes and shapes which can range between about 1 nm and 1000 nm with a variety of area to volume ratios.
“Metal layer” means the thin metal (e.g., gold) surface or metal (e.g., gold) nanoparticle on the SPR or LSPR sensor substrate.
“NH₂-PEG-COOH” means a heterobifunctionalized PEG polymer with an amino group on one end and a carboxylic acid functional group on the other end.
“Nitrogen containing functional group” means any primary, secondary, tertiary or quaternary amine or amino group.
“Peptide Spacer” means a sequence of amino acids (e.g., Glycine) which will be used as the spacer between active amino acids having extra carboxylic acid functions. The number of repeating units in the structure of peptide spacers vary based on required spaces between active amino acids. The type of building blocks in the structure of peptide spacers may be different based on the desired hydrophilicity. Peptide spacers could be made by repeating the same unit (e.g., several Glycine units) or by combination of different building blocks.
“Polyalkylene glycol” or “PAG” means a substituted or unsubstituted linear or branched carbon chain incorporating one or more alkalene glycol moieties (i.e., oxyalkalene moieties), and optionally incorporating one or more additional moieties selected from the group consisting of —S—, —O—, —N—, and —C(O)—. Examples include polyethylene glycol (PEG), polyoxyethylene (POE), polypropylene glycol (PPG), polybutylene glycol (PBG), amine-PEG-carboxyl (NPC) and combinations thereof (e.g., linear or branched polymers including combinations of two or more different PAG subunits, such as two or more different PAG units selected from PEG, PPG, PBG and NPC subunits).
“Sensor” means the combination of the “sensor substrate” and the “metal layer” used for SPR and LSPR spectroscopy.
“Sensor substrate” means the silica containing (e.g., glass) layer on which the remaining portion of the SPR and LSPR sensors are held.
“Spacer” means a chemical located in the linker body used to lengthen or shorten the chemical (e.g., peptide) chain.
“Spacer amino acid” means any natural or custom synthesized amino acid used as a spacer in the linker.
“Spacer group” means any natural or synthesized molecule which acts as a Spacer in the linker.
“Sulfur containing functional group” means any sulfur containing molecule.

### NTA Linkers

As NTA linkers are multi-functional and need specific spatial orientation to provide effective and stable chelation properties, their synthesis could be challenging. Despite this complexity, here we are reporting a robust synthetic method for production of NTA linkers with unlimited structural flexibility in solid phase. The flexibility of this method allows us to synthesize one or more NTA functional groups combined with other different functional groups and spacers to provide desired biochemical properties. One terminus of the linker can be chemisorbed on a gold surface, for example via thiol or amino groups. The distal end, or head, of the linker is one or more NTA groups. Between these termini, a variety of different spacer groups are possible ranging from one or more hydrophobic-CH₂-sequences to one or more hydrophilic PEG chains both with a tunable number of units.

Furthermore, two or more NTA functional groups are possible on a single linker molecule in which the length of each NTA branch could be equal or asymmetric (having arms with unequal length). This later case could be important when an immobilized protein is big in size and the immobilization is limited by steric hindrance. This type of linker allows for different levels of immobilization for maximum efficiency in the range of decay length.

### SPR Sensor

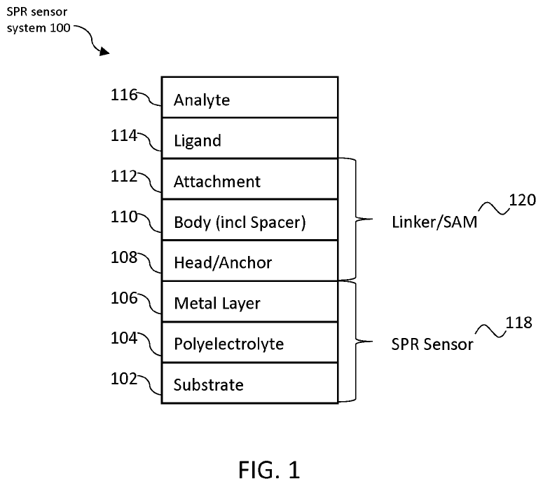

In some embodiments, as shown in FIG. 1, the SPR sensor assembly 100 consists of an SPR sensor 118 with a self-assembled monolayer (SAM) of a chemical linker 120 which may bind with a ligand 114 which captures analyte molecules 116. The SPR sensor 118 consists of a silica containing substrate 102 functionalized with a polyelectrolyte 104. In some embodiments, the substrate may be selected from silicon containing substrates, glass substrates, polystyrene substrates and agarose substrates. In some embodiments, the silica containing substrate 202 may be silicon chips, glass slides, polystyrene beads, controlled pore glass beads, hybrid controlled pore glass beads, agarose beads, micro-beads and micro-spheres. It may be appreciated by one of skill in the art that numerous other silica containing substrates may be used. In some embodiments, the polyelectrolyte 104 changes the surface charge of the silica containing substrate 102 which allows binding of various metals of the same charge as the silica containing substrate 102. In some embodiments, the functionalized silica substrate 104 of the SPR sensor 118 is coated with a thin metal layer 106. The thin metal layer 106 together with the polyelectrolyte 104 functionalized silica containing substrate 102 create the SPR sensor 118. In another embodiment, the thin metal layer 106 is coated with a chemical layer 120 comprised of a self-assembled monolayer (SAM) of peptide linkers. In some instances, the thin metal layer 106 may comprise metal nanoparticles. The metal nanoparticles may be configured to produce an optical signal.

In some embodiments, the SPR sensor chip 118 may be a transparent glass substrate, for example, coated with the remaining components of an SPR sensor. In some embodiments the transparent glass substrate 102 is functionalized with a polyelectrolyte 104 to allow the metal layer 106 to electrostatically attach to the sensor substrate. In another embodiment the polyelectrolyte 104 is poly(allylamine hydrochloride) (PAH) for example. PAH may be purchased from Sigma-Aldrich (MilliporeSigma, St. Louis, MO, USA) as an example. In another embodiment, the SPR layer 106 may, for example, be a gold layer 106. The gold layer 106 may be chemically modified with a linker 120 to enable the immobilization of one of the binding partners such as a ligand 114. The components of the linker 120 are discussed below. In some embodiments, the gold layer 106 may be from about 1 nm to about 1000 nm thick. Gold coated transparent glass slides may be purchased from GE Healthcare (Uppsala. Sweden) for example.

### LSPR Sensor

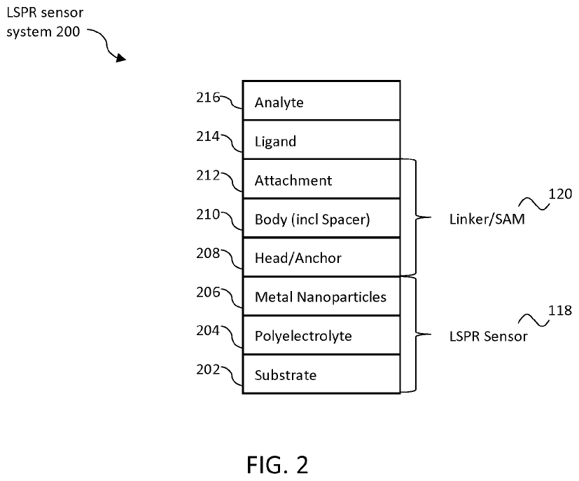

In some embodiments, as shown in FIG. 2, the LSPR sensor system 200 consists of an LSPR sensor 218 with a self-assembled monolayer (SAM) of a chemical linker 220 which may bind with a ligand 214 which captures analyte molecules 216. The LSPR sensor 218 consists of a silica containing substrate 202 functionalized with a polyelectrolyte 204. In some embodiments, the substrate 202 may be selected from silicon containing substrates, glass substrates, polystyrene substrates and agarose substrates. In some embodiments, the silica containing substrate 202 may be silicon chips, glass slides, polystyrene beads, controlled pore glass beads, hybrid controlled pore glass beads, agarose beads, micro-beads and micro-spheres. It may be appreciated by one of skill in the art that numerous other silica containing substrates may be used. In some embodiments, the polyelectrolyte 204 changes the surface charge of the silica containing substrate 202 which allows binding of various metals having the same charge as the silica containing substrate 102. In some embodiments, the functionalized silica substrate 204 of the LSPR sensor 218 is coated with adsorbed metal nanoparticles 206. The layer of adsorbed metal nanoparticles 206 together with the polyelectrolyte 204 functionalized silica containing substrate 202 create the LSPR sensor 218. In another embodiment, the metal nanoparticles 206 are coated with a chemical layer 220 comprised of a SAM of peptide linkers.

In some embodiments, the LSPR sensor chip 218 may be a transparent glass substrate, for example, coated with the remaining components of an LSPR sensor. In some embodiments the transparent glass substrate 202 is functionalized with a polyelectrolyte 204 to allow the metal nanoparticles to electrostatically attach to the sensor substrate. In another embodiment the polyelectrolyte 204 is poly(allylamine hydrochloride) (PAH) for example. In another embodiment, the metal nanoparticles 206 may be gold nanoparticles 206. The gold nanoparticles 206 may be chemically modified with a linker 220 to enable the immobilization of one of the binding partners such as a ligand 214. The components of the linker 120, 220 are discussed below. In some embodiments, the dimensions of the metal nanoparticles can range between about 1 nm and 1000 nm with a variety of area to volume ratios. The gold nanoparticles may be purchased commercially, for example from Sigma-Aldrich (MilliporeSigma, St. Louis, MO) or NanoComposix (San Diego, CA, USA).

Compositions of metal layers and metal nanoparticles that can be used for SPR and LSPR include gold, silver, platinum, gold coated silver, silver coated gold, combinations of these metals, and others. The shape of the nanoparticles used can also vary. Useful nanoparticle shapes include but are not limited to, rods, stars, urchins, decahedra, hexagons, triangles, shells, prisms, platelets, spheres, rice, plates, cubes, cages, and bipyramids.

### Linker

The linkers of the disclosure typically include a head (or “anchoring site”) 108 for coupling to the SPR or LSPR substrate and a spacer 110. The linker 120 includes one or more attachment sites 112 on the distal end or ends of the spacer 110 (or “body”) which couple to a ligand 114. Distal may mean a terminal end of the spacer or a position away from the substrate surface or sensor surface. In some embodiments, the linker 120 may comprise a matrix. In some embodiments, the linker 120 may be disposed over a portion of a surface of the metal nanoparticle 106. Although it will be appreciated that a wide variety of variations and alternative embodiments could be conceived or constructed by those skilled in the art based on the teachings of the present disclosure, the linker-ligand structure may be simply illustrated as follows:
Substrate 106—head 108—spacer 110—attachment 112—ligand 114.

In operation, the ligand may couple to an analyte:
Substrate 106—head 108—spacer 110—attachment 112—ligand 114—analyte 116.

The resulting combination may be detected using SPR or LSPR spectroscopy.

It will be appreciated that a wide variety of combinations of chemicals are possible within the scope of the disclosure to optimize the length, steric considerations, hydrophobicity/hydrophilicity, lipophilicity/lipophobicity and density of attachment sites 112 in the linker. In some embodiments, the arrangement of chemicals in the tunable linker 120 may be represented by the following schematic:
HG-SG-AG;
wherein “HG” is the ‘head group,” “SG” is the “spacer group,” and “AG” is the “attachment group.” In another embodiment, there may be multiple head 108 groups attached in succession (e.g., linear) or branched fashion in the tunable linker 120. In another embodiment, there may be multiple spacer 110 groups attached in succession or in a branched fashion in the tunable linker 120. In another embodiment both the head 108 and spacer 110 groups may be arranged in a combination of linear or branched arrangements. In another embodiment there may be multiple repeating units of -SG-AG in the tunable linker. These embodiments may be represented by the following schematics:
HG_{X}-SG_{Y}-AG_{A}; and
HG_{X}[-SG_{Y}-AG_{A}]_{Z};
wherein the arrangement of HG, SG and AG may be in a linear or branched chain arrangement; and wherein there are always the appropriate number of bonds between chemical groups; wherein X represents the number of head 108 groups arranged in succession or any other arrangement such as branched chain in the head 108 region of the linker 120; and wherein Y represents the number of spacer 110 groups arranged in succession or any other arrangement such as branched chain in the spacer 110 region of the linker 120; and wherein A represents the number of attachment groups 112 arranged in succession or any other arrangement such as branched chain in the spacer region 110 of the linker 120; and wherein Z represents the number of repeating -SG_{Y}-AG_{A} groups arranged in succession or any other arrangement such as branched chain in the spacer 110 region of the linker 120; and wherein X ranges from 1 to 20, or from 1 to 8, or from 1 to 6 or from 1 to 4; and Y ranges from 0 to 50, or from 0 to 40, or from 0 to 30, or from 0 to 20, or from 0 to 10 or from 0 to 5; and A ranges from 1 to 50, or from 1 to 40, or from 1 to 30, or from 1 to 20 or from 1 to 10, or from 1 to 5; and Z may range from 1 to 10, or from 2 to 8, or from 2 to 6, or from 2 to 4. In some embodiments, the linker may be a matrix of a biopolymer, such as a polysaccharide, a polysaccharide derivative, a polymer, a functionalized polymer, a protein, or a combination thereof.

In some embodiments, the arrangement of the chemicals in the linker 120 are arranged in the following order: HG-SG-AG-SG-AG-SG-AG-SG-AG. In this embodiment. Z is equal to 4 as an example shown for clarity. In some embodiments, the linker head 108, spacer 110 and attachment 112 groups may all be composed of peptides. In this embodiment the linker is a tunable peptide linker. In some embodiments, the linker head 108, spacer 110, and attachment 112 groups may each be composed of saccharides.

### Linker-Head

In some embodiments, as shown in FIG. 1, the linker head 108 or the linker “anchoring site” may be composed of any chemical functional group which binds to the substrate 106. In another embodiment, the linker head 108 may be composed of a sulfur containing chemical group. In another embodiment, the linker head 108 may be composed of a sulfide (R—S—R′) chemical group. In another embodiment, the linker head 108 may be composed of a thiol (—SH) chemical group. In another embodiment, the linker head 108 may be composed of a disulfide (—S—SH) chemical group. In another embodiment, the linker head 108 may be composed of any functional group with more than two sulfurs. In another embodiment, the linker head 108 may be composed of any group VI chalcogen.

In another embodiment, the linker head 108 may be composed of at least one methionine amino acid group, which includes a sulfide group. In another embodiment, the linker head 108 may be composed of at least one cysteine amino acid group, which includes a thiol group. Binding occurs spontaneously between a gold substrate 106 and thiol functional groups. Binding occurs spontaneously between a gold substrate 106 and sulfide functional groups.

In another embodiment, the linker head 108 may be composed of at least one thiol bound to a saccharide. In another embodiment, the linker head 108 may be composed of at least one thiolglucose. Binding occurs spontaneously between a gold substrate 106 (e.g., a Au NP) and sulfide functional groups, such that covalent bond(s) is/are formed.

In another embodiment, the linker head 108 may be composed of a carbon containing chemical group. In another embodiment, the linker head 108 may be composed of a diazonium salt. Binding occurs spontaneously between a gold substrate 106 and diazonium functional groups.

In another embodiment, the linker head 108 may be composed of a nitrogen containing chemical group. In another embodiment, the linker head 108 may be composed of an amino group. In another embodiment, the linker head 108 may be composed of a primary, secondary, tertiary or quaternary amino group. Binding occurs spontaneously between a gold surface and amino functional groups.

In another embodiment, the linker head 108 may be composed of any chemical functional group which binds readily to any metal (e.g., gold) surface 106.

In some embodiments, the HG is an amino acid. In this embodiment, the HG is a head amino acid (HAA) composed of any amino acid whether natural or synthetic. The arrangement may be shown more generally by the following schematics:
HAA_{X}-SG_{Y}-AG_{A}; and
HAA_{X}[-SG_{Y}-AG_{A}]_{Z};
wherein the arrangement of HAA, SG and AG may be in a linear or branched chain arrangement; and wherein there are always the appropriate number of bonds between chemical groups; and wherein X represents the number of HAA groups arranged in succession or any other arrangement such as branched chain in the head 108 region of the linker 120, and wherein Y represents the number of spacer 110 groups arranged in succession or any other arrangement such as branched chain in the body 110 region of the linker 120, and wherein A represents the number of attachment groups 112 arranged in succession or any other arrangement such as branched chain in the body region 110 of the linker 120, and wherein Z represents the number of repeating -SG_{Y}-AG_{A} groups arranged in succession or any other arrangement such as branched chain in the body 110 region of the linker 120, and wherein X ranges from 1 to 20, or from 2 to 8, or from 2 to 6 or from 2 to 4 and Y ranges from 0 to 50, or from 0 to 40, or from 0 to 30, or from 0 to 20, or from 0 to 10, or from 0 to 5 and A ranges from 1 to 50, or from 1 to 40, or from 1 to 30, or from 1 to 20 or from 1 to 10, or from 1 to 5; and Z may range from 1 to 10, or from 2 to 8, or from 2 to 6, or from 2 to 4.

In another embodiment, the linker head 108 is at least one cysteine amino acid group, which can be represented by the following schematics:
Cys_{X}-SG_{Y}-AG_{A}; and
Cys_{X}[-SG_{Y}-AG_{A}]_{Z};
wherein X represents the number of Cys groups in the linker head 108. In an embodiment the number of cysteine amino acids in the linker head 108 ranges from 1 to 20 or from 1 to 10 or from 1 to 5. In a preferred embodiment, the number of cysteine amino acids at the linker head is 1.

In an embodiment, the head of the linker is at least one methionine amino acid, which can be represented by the following schematics:
Met_{X}-SG_{Y}-AG_{A}; and
Met_{X}[-SG_{Y}-AG_{A}]_{Z};
wherein X is the number of Met groups in the linker head. In an embodiment the number of methionine amino acids in the linker head range from 1 to 20 or from 1 to 10 or from 1 to 5. In a preferred embodiment, the number of methionine amino acids at the linker head is 1.

In an embodiment, the head of the linker is composed of at least one sulfur containing functional group “SR,” which may be represented by the following schematic:
SR_{X}-SG_{Y}-AG_{A}; and
SR_{X}[-SG_{Y}-AG_{A}]_{Z};
wherein X represents the number of sulfur containing “SR” functional groups in the linker head. The sulfur containing functional groups, “SR,” include any molecule either naturally occurring or synthetic which contains one or more sulfur elements in a linear or branched chain configuration such as disulfides, dithiocarbamates, dithiocarbamic acids, thiocarboxylic acids (thioacids), carbothioic acids or the anions of any of these molecules. In an embodiment the number of sulfur containing functional groups in the linker head ranges from 1 to 20 or from 1 to 10 or from 1 to 5. In a preferred embodiment, the number of sulfur containing functional groups at the linker head is 1.

In an embodiment, the head of the linker is composed of at least one diazonium functional group which may be represented by the following schematic:
Da_{X}-SG_{Y}-AG_{A}; and
Da_{X}[-SG_{Y}-AG_{A}]_{Z};
wherein X represents the number of diazonium functional groups in the linker head. In an embodiment the number of diazonium functional groups in the linker head ranges from 1 to 20 or from 1 to 10 or from 1 to 5. In a preferred embodiment, the number of diazonium functional groups at the linker head is 1.

In another embodiment, the head of the linker is composed of at least one amino functional group, including primary, secondary, tertiary and quaternary amino groups, “NR,” which may be represented by the following schematic:
NR_{X}-SG_{Y}-AG_{A}; and
NR_{X}[-SG_{Y}-AG_{A}]_{Z};
wherein X represents the number of primary, secondary, tertiary or quaternary amino “NR” functional groups in the linker head. In an embodiment the number of amino functional groups in the linker head ranges from 1 to 20 or from 1 to 10 or from 1 to 5. In a preferred embodiment, the number of amino functional groups at the linker head is 1.

The thiol (—SH) functional group in cysteine, the sulfide (R—S—R′) functional group in methionine, and the diazonium functional group (R—N₂⁺) bind spontaneously to the gold substrate or nanoparticle. Increasing the number of thiol functional groups by increasing the number. X. of cysteines in the tunable peptide linker head causes a stronger linker attachment, or anchor, to the gold. Increasing the number of sulfide functional groups by increasing the number. X. of methionines in the tunable peptide linker head causes a stronger linker attachment to the gold. Increasing the number of other sulfur containing functional groups in the tunable peptide linker head causes a stronger linker attachment to the gold. Increasing the number of diazonium functional groups in the tunable peptide linker head causes a stronger linker attachment to the gold. Increasing the number of amino functional groups in the tunable peptide linker head causes a stronger linker attachment to the gold. Increasing the number of carbon containing functional groups in the tunable peptide linker head causes a stronger linker attachment to the gold. In an embodiment of the disclosure, the stability of the peptide linker SAM attached to the metal surface is therefore tunable by selecting the number of head functional groups which bind to the (e.g., gold) substrate.

In some embodiments, the thiol (—SH), the sulfide (R—S—R′), and diazonium (R—N₂⁺) functional groups bind spontaneously to the metal substrate (e.g., Au substrate) or nanoparticle (e.g., AuNP). Increasing the number of thiol, sulfide, or diazonium groups of the head saccharide (HS) may increase the density of saccharides on a surface of the metal nanoparticle or substrate.

### Linker-Body/Spacer

The characteristics of the linker “body” 110 or linker mid-section may be adjusted for length, steric considerations, and density of attachment 112 sites for the ligand 114. Although it will be appreciated that a wide variety of variations could be conceived or constructed by those skilled in the art, the linker body 110 may be configured in a linear (e.g., “straight chain”) fashion. Additionally, the linker body 110 may be configured with one or more branched chains in the chemical structure. The linker body 110 may include a molecule which acts as a “spacer” 110.

In some embodiments, a spacer 110 molecule may be any molecule whose purpose is to increase or decrease the length of the linker 120. In some embodiments, the spacer 110 may be any molecule chosen based on its length. For example, the spacer 110 may be a polymer. In this embodiment, the number of repeating monomer units may be selected to increase or decrease the length of the spacer 110. In another embodiment, the spacer 110 may be a monomer unit of a polymer. Although it will be appreciated that a wide variety of variations could be conceived or constructed by those skilled in the art, the spacer 110 may be any variety of polyalkylene glycol (PAG) or any functionalized variety of PAG such as NH₂-PEG-COOH (NPC).

PAG compounds such as PEG are commercially available from a variety of sources, such as Sigma-Aldrich (MilliporeSigma, St. Louis, MO) and BroadPHARM (San Diego, CA) for example. Monodisperse PEG is preferred. In another embodiment, the spacer molecules may be “NH₂-PEG-COOH” (NPC), commercially available from Sigma-Aldrich (MilliporeSigma, St. Louis, MO) and Advanced Biochemicals (Lawrenceville, GA) for example. Monodisperse NPC is preferred.

In some embodiments, the spacer 110 may be any natural or synthetic amino acid. In this embodiment, the amino acid acting as a spacer 110 may be a “spacer amino acid” (SAA). The number of SAAs connected together may be selected to increase or decrease the length of the spacer 110. In some embodiments, the SAAs may be the same amino acid or a combination of any natural or synthetic amino acid. In another embodiment, the number of SAAs varies from 0 to 50 or from 0 to 40 or from 0 to 30 or from 0 to 20 or from 0 to 10 or from 0 to 5. In a preferred embodiment, the number of SAAs is 1.

In some embodiments, the SAA may be composed of glycine. In another embodiment, the SAA may be composed of phenylalanine. In another embodiment, the SAA may be any amino acid or a sequence of two or more amino acids which are used to increase or decrease the length of the linker. In some embodiments, the spacer may be composed of repeating units of any one of the foregoing molecules. In another embodiment, the spacer or SAA may be composed of a wide variety of combinations of molecules, polymers, monomers, amino acids or other chemicals whether naturally occurring, commercially available or synthesized in the laboratory.

The amino acids used in the linkers in the disclosure may be purchased from suppliers or manufactured manually via solid-phase peptide synthesis (SPPS) described below or using a peptide synthesis machine. Examples of suitable machines include CSBio II from CSBio (Menlo Park, California) or Biotage® Initiator+Alstra™ Peptide Synthesizer from Biotage (Uppsala, Sweden). Examples of suppliers who provide custom synthesized peptide strands include Biomatik (Wilmington, DE) and Thermo Fisher Scientific (Waltham, MA).

In some embodiments, the spacer 110 may be any natural or synthetic saccharide. In this embodiment, the saccharide acting as a spacer 110 may be a “spacer saccharide” (SS). The number of SSs connected together may be selected to increase or decrease the length of the spacer 110. In some embodiments, the SSs may be the same amino acid or a combination of any natural or synthetic amino acid. In another embodiment, the number of SSs varies from 0 to 50 or from 0 to 40 or from 0 to 30 or from 0 to 20 or from 0 to 10 or from 0 to 5. In some embodiments, the number of SSs is 1. In some embodiments, the number of SSs is 0.

In some embodiments, the SAA may be composed of glycine. In another embodiment, the SAA may be composed of phenylalanine. In another embodiment, the SAA may be any amino acid or a sequence of two or more amino acids which are used to increase or decrease the length of the linker. In some embodiments, the spacer may be composed of repeating units of any one of the foregoing molecules. In another embodiment, the spacer or SAA may be composed of a wide variety of combinations of molecules, polymers, monomers, amino acids, saccharides, or other chemicals whether naturally occurring, commercially available or synthesized in the laboratory.

The amino acids used in the linkers in the disclosure may be purchased from suppliers or manufactured manually via solid-phase peptide synthesis (SPPS) described below or using a peptide synthesis machine. Examples of suitable machines include CSBio II from CSBio (Menlo Park, California) or Biotage® Initiator+Alstra™ Peptide Synthesizer from Biotage (Uppsala, Sweden). Examples of suppliers who provide custom synthesized peptide strands include Biomatik (Wilmington, DE) and Thermo Fisher Scientific (Waltham, MA).

The saccharides used in the linkers in the disclosure may be purchased from suppliers. The saccharides may be chemically modified as described below.

In some embodiments, both the HG and SG are amino acids. In another embodiment, the HG is a head amino acid (HAA) composed of any amino acid whether natural or synthetic and the SG is a spacer amino acid (SAA) composed of any amino acid whether natural or synthetic. The arrangement may be shown more generally by the following schematics:
HAA_{X}-SAA_{Y}-AG_{A}; and
HAA_{X}[-SAA_{Y}-AG_{A}]_{Z};
wherein the arrangement of HAA, SAA and AG may be in a linear or branched chain arrangement; and wherein there are always the appropriate number of bonds between chemical groups; and wherein X represents the number of HAA groups arranged in succession or any other arrangement such as branched chain in the head region of the linker; and wherein Y represents the number of SAA groups arranged in succession or any other arrangement such as branched chain in the body region of the linker; and wherein A represents the number of attachment groups 112 arranged in succession or any other arrangement such as branched chain in the body region 110 of the linker 120; and wherein Z represents the number of repeating -SAA_{Y}-AG_{A} groups arranged in succession or any other arrangement such as branched chain in the body region of the linker; and wherein X ranges from 1 to 20, or from 2 to 8, or from 2 to 6, or from 2 to 4; and Y ranges from 0 to 50, or from 0 to 40, or from 0 to 30, or from 0 to 20, or from 0 to 10, or from 0 to 5 and A ranges from 1 to 50, or from 1 to 40, or from 1 to 30, or from 1 to 20 or from 1 to 10, or from 1 to 5; and Z may range from 1 to 10, or from 2 to 8, or from 2 to 6, or from 2 to 4.

In another embodiment, the linker spacer is at least one glycine amino acid group, which can be represented by the following schematic:
HAA_{X}-Gly_{Y}-AG_{A}; and
HAA_{X}[-Gly_{Y}-AG_{A}]_{Z};
wherein Y represents the number of Gly groups in the linker body. In an embodiment the number of glycine amino acids in the linker body ranges from 1 to 20. In a preferred embodiment, the number of glycine amino acids at the linker body is 1.

In some embodiments, each of HG, SG, and AG are saccharides. In another embodiment, the HG is a saccharide composed of a modified saccharide comprising SR¹, NR¹R², OR¹, CR¹, wherein R¹ and R² are each independently hydrogen, halogen, C₁-C₁₀ alkyl, C₁-C₁₀ alkenyl, C₁-C₁₀ alkynyl, nitrile, C₁-C₁₀ carbocycle, C₁-C₁₀ aryl, C₁-C₁₀ heteroaryl, C₁-C₁₀ heterocycle, or a combination thereof. The arrangement may be shown more generally by the following schematics:
HG_{X}-SG_{Y}-AG_{A}; and
HG_{X}[-SG_{Y}-AG_{A}]_{Z};
wherein the arrangement of HG, SG, and AG may be in a linear or branched chain arrangement; and wherein there are the appropriate number of bonds between chemical groups; and wherein X represents the number of HAA groups arranged in succession or any other arrangement such as branched chain in the head region of the linker. Y represents the number of SG groups arranged in succession or any other arrangement such as branched chain in the body region of the linker. A represents the number of attachment groups arranged in succession or any other arrangement such as branched chain in the body region of the linker. Z represents the number of repeating -SG_{Y}-AG_{A} groups arranged in succession or any other arrangement such as branched chain in the body region of the linker. X may range from 1 to 20, or from 2 to 8, or from 2 to 6, or from 2 to 4; and Y ranges from 0 to 50, or from 0 to 40, or from 0 to 30, or from 0 to 20, or from 0 to 10, or from 0 to 5 and A may range from 1 to 50, or from 1 to 40, or from 1 to 30, or from 1 to 20 or from 1 to 10, or from 1 to 5; and Z may range from 1 to 10, or from 2 to 8, or from 2 to 6, or from 2 to 4.

In another embodiment, the linker spacer is at least one dextrose group, which can be represented by the following schematic:
HG_{X}-[dextrose]_{Y}-AG_{A}; and
HG_{X}[-[dextrose]_{Y}-AG_{A}]_{Z};
wherein Y represents the number of dextrose groups in the linker body. In some embodiments, the number of dextrose groups in the linker body ranges from 1 to 20. In some embodiments, the number of spacer saccharides (SS) at the linker body is 1. In some embodiments, the number of spacer saccharides (SSs) at the linker body is 0 (i.e., Y is 0). Each of the dextrose units may be bound to another dextrose unit via a 1,1-, 1,2-, 1,3-, 1,4-, 1,5-, or 1,6-glycosidic bond.

In another embodiment, the HG is a head amino acid (HAA) composed of any amino acid whether natural or synthetic and the SG is a “polyalkylene glycol” (PAG) such as polyethylene glycols, polypropylene glycols, amine-PEG-carboxyls (NPC), etc., and various combinations of the forgoing. The arrangement may be shown more generally by the following schematics:
HAA_{X}-PAG_{Y}-AG_{A}; and
HAA_{X}[-PAG_{Y}-AG_{A}]_{Z};
wherein the arrangement of HAA, PAG and AG may be in a linear or branched chain arrangement; and wherein there are the appropriate number of bonds between chemical groups; and wherein X represents the number of HAA groups arranged in succession or any other arrangement such as branched chain in the head region of the linker; and wherein Y represents the number of PAG groups arranged in succession or any other arrangement such as branched chain in the body region of the linker; and wherein A represents the number of attachment groups 112 arranged in succession or any other arrangement such as branched chain in the body region 110 of the linker 120; and wherein Z represents the number of repeating -PAG_{Y}-AG_{A} groups arranged in succession or any other arrangement such as branched chain in the body region of the linker; and wherein X ranges from 1 to 20, or from 2 to 8, or from 2 to 6 or from 2 to 4 and Y ranges from 0 to 50, or from 0 to 40, or from 0 to 30, or from 0 to 20, or from 0 to 10, or from 0 to 5 and A ranges from 1 to 50, or from 1 to 40, or from 1 to 30, or from 1 to 20, or from 1 to 10, or from 1 to 5; and Z may range from 1 to 10, or from 2 to 8, or from 2 to 6, or from 2 to 4.

In some embodiments, the number of PAG units in the spacer, Y, ranges from 1 to about 20. In another embodiment, the number of PAG units in the spacer, Y, ranges from about 2 to about 15. In another embodiment, the number of PAG units in the spacer, Y, ranges from about 2 to about 10. In another embodiment, the number of PAG units in the spacer, Y, is about 8. In some embodiments, the PAG units comprise PEG units and the number of PEG units in the spacer, Y, ranges from 1 to about 20. In another embodiment, the PAG units comprise PEG units and the number of PEG units in the spacer, Y, ranges from about 2 to about 15. In another embodiment, the PAG units comprise PEG units and the number of PEG units in the spacer, Y, ranges from about 2 to about 10. In another embodiment, the PAG units comprise PEG units and the number of PEG units in the spacer, Y, is about 8. In a preferred embodiment, PAG is NPC. In some embodiments, the number of NPC units in the spacer, Y, ranges from 1 to about 20. In another embodiment, the number of NPC units in the spacer, Y, ranges from about 2 to about 15. In another embodiment, the number of NPC units in the spacer, Y, ranges from about 2 to about 10. In another embodiment, the number of NPC units in the spacer, Y, is about 8. In another preferred embodiment, PAG is NPC and Y is 8.

In an embodiment of the disclosure, the length of the linker is tunable. In some embodiments the length of the peptide linker is tunable by varying the number and type of spacer groups.

In another embodiment of the disclosure, the head group (HG) is a functional group composed of any group whether natural or synthetic saccharide. In some embodiments, the HG may be a saccharide. In some embodiments, the saccharide may be a polysaccharide. In yet other embodiments, the saccharide may be a disaccharide. In some instances, the saccharide may comprise carboxymethyldextrose, dextrose, amylose, galactose, or a combination thereof. In some embodiments, the saccharide may be thiolated. In some instances, the headgroup may comprise a disaccharide.

### Linker-Attachment Site

The linker 120 may also include a molecule or functional group located at distal ends of the linear or branched molecular chain linker 120 which acts as an attachment site 112 for the ligand. The linkers 120 may use one or more attachment sites 112 to couple to a ligand 114 functional group.

In some embodiments, the linker attachment site 112 is a molecule composed of any chemical functional group which binds to the ligand 114. In some embodiments, the linker attachment site 112 contains any chemical functional group which connects to the previous molecule in the linker chemical chain. In some embodiments, the linker attachment site 112 contains any chemical functional group which connects to the subsequent molecule in the linker 120 chemical chain. In some embodiments, the linker attachment site 112 may further comprise a metal ion. In some embodiments, the metal ion comprises a transition metal ion. In yet other embodiments, the transition metal ion comprises nickel, iron, cobalt, or copper. In some embodiments, the metal ion is selected from the group consisting of nickel, iron, cobalt, copper, and calcium. In some instances, the metal ion may be configured to chelate to aromatic side chains of proteins (e.g., histidines, tyrosines, tryptophans, phenylalanines) and to the attachment site 112, such as attachment sites comprising carboxylates.

In some embodiments there are one or more functional groups at the linker attachment site 112. In a preferred embodiment, the linker attachment site is composed of three functional groups. In a most preferred embodiment, two functional groups at the linker attachment site are used to connect to the previous and subsequent molecules in the chain and the third functional group is used to provide the attachment site 112 for the ligand 114. In a preferred embodiment, the one or more chemical functional groups contain a carboxylic acid functional group. In a preferred embodiment, the one or more chemical functional groups contain an amino functional group. In some embodiments, the attachment site 112 is an amino acid. In a preferred embodiment the attachment site 112 is a nitrilotriacetic acid (NTA) moiety. If the attachment site is located on an amino acid, this amino acid is called an “attachment amino acid” (AAA). Examples of attachment amino acids (AAAs) that may be used in the linkers 120 of the disclosure include: aspartic acid, glutamic acid, any natural or synthesized amino acid having an extra functional group (e.g., lysine with an extra amino group) and mixtures of any of the foregoing. If the attachment site is located on a saccharide, this saccharide is called an “attachment saccharide” (AS). In some embodiments, any molecule which binds with a ligand 114 such as biotin or nitrilotriacetic acid may be attached to the linker body 110 to become an attachment site 112.

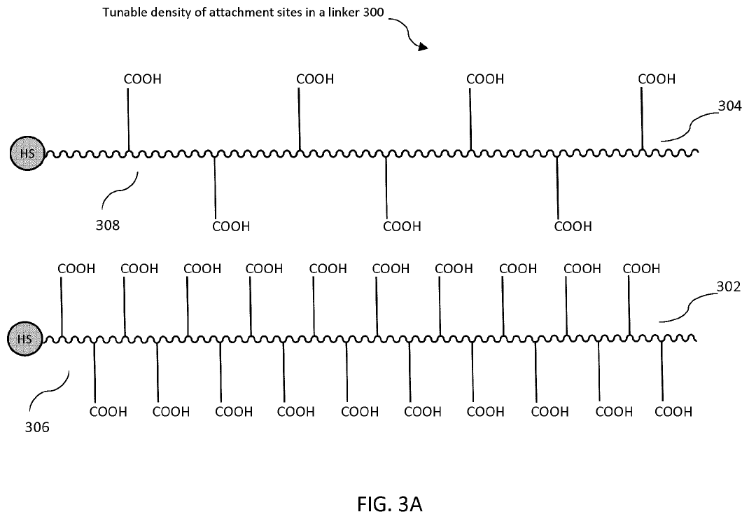

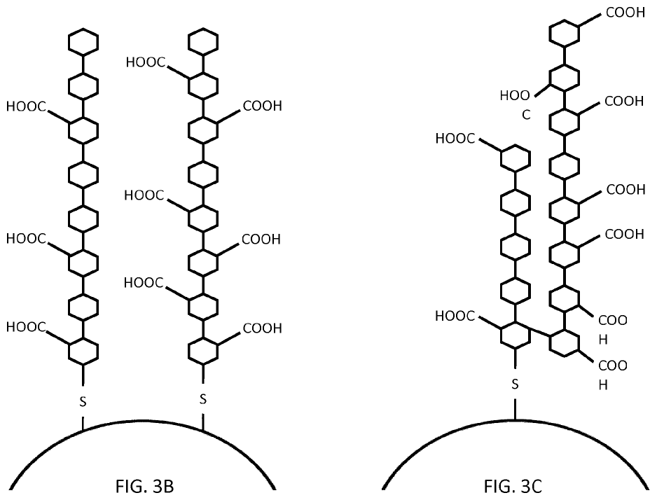

The characteristics of the spacer 110 molecule may be tuned to adjust the length of the linker 120 and the density of attachment sites 112. The length of the linker 120 and the density of attachment sites 112 are tunable based on the steric considerations of the ligand 114 and the analyte. In some embodiments, the attachment amino acid (AAA) may be aspartic acid which has one carbon spacer between attachment functional groups. In another embodiment, the AAA may be glutamic acid which has two carbon spacers between attachment functional groups. In another embodiment the AAA may be lysine which has four carbon spacers between attachment functional groups. In another embodiment, the spacers may be a mix of AAAs. In another embodiment, the number of AAAs and the particular sequence of AAAs may be chosen to tune the length of the linker 120 and to tune the density of attachment sites 112. FIG. 3 illustrates exemplary SPR or LSPR tunable linker molecules with a decreased or increased density of attachment sites (e.g., —COOH) and a conversely increased or decreased number of spacer molecules, represented by wavy lines. As seen in FIG. 3, creating a linker 120 with more aspartic acid moieties for example may create a higher density of attachment sites 302, whereas creating a linker with more lysine moieties for example may create a lower density of attachment sites 304 simply because the increased number of —CH₂-units creates a larger distance between subsequent carboxylic acid (for example) attachment moieties.

Increasing the number of ligand 114 moieties coupled to the linker 120 increases the number of binding sites available for capturing analyte 116 molecules. Increasing the number of captured analyte 116 molecules results in an improvement in the SPR and LSPR signal.

In some embodiments, the attachment saccharide (AS) may be carboxymethyl dextrose (CMD) which has a carboxylate group. In another embodiment, the AS may be carboxymethyldextrose (CMD) which has been modified with at least one polydentate group. In some embodiments, the polydentate group comprises one or more carboxylic acid groups. In some embodiments, the polydentate group comprises nitrilotriacetic acid (NTA), tris-(2-aminoethyl)amine, or a combination thereof. In some embodiments, the polydentate group is a tridentate group. In some embodiments, the polydentate group is nitrilotriacetic acid (NTA), which has three carboxylate groups. In another embodiment, the spacers may be a mix of saccharides. In another embodiment, the number of ASs may be chosen to tune the length of the linker 120 and to tune the density of attachment sites 112, such as the density of available NTA sites. FIG. 3B illustrates exemplary SPR or LSPR tunable linker molecules with a decreased or increased density of attachment sites (e.g., —COOH) and a conversely increased or decreased number of spacer molecules, represented by hexagons (e.g., for a polysaccharide backbone). As seen in FIG. 3B, creating a linker 120 with more —COOH moieties (e.g., through incorporating NTA) may create a higher density of attachment sites 302. As illustrated in FIG. 3C, creating a linker 120 with more carboxylate moieties bound to the sugars (hexagons) that are bound to other saccharide chains via glycosidic bonds may create a higher density of attachment sites for attaching ligands. In some embodiments, the linker (e.g., a matrix) may comprise a density of attachment sites (e.g., polydentate sites) that may range from about 1% to about 100%, from about 10% to about 90%, from about 20% to about 80%, from about 25% to about 75%, from about 30% to about 70%, or from about 40% to about 60% by volume. In some embodiments, the density of attachment sites may range from about 1% to about 100%, from about 10% to about 90%, from about 20% to about 80%, from about 25% to about 75%, from about 30% to about 70%, or from about 40% to about 60% of the total number of available sites of the linker. In some embodiments, the density of attachment sites is at least about 25% by volume. In some embodiments, the density of attachment sites is at least about 25% of the total number of available sites. In some embodiments, the density of attachment sites is no more than about 90%. In some embodiments, the density of attachment sites is no more than about 90% of the total number of available sites.

The linker 120 may be selected or designed to avoid capturing non-target molecules in the sample thereby reducing non-specific binding (NSB) noise below the current state of the art. Increasing the signal and reducing the noise improves the signal to noise ratio of the SPR and LSPR signal.

In some embodiments, the HG, SG and AG are amino acids. In another embodiment, the HG is a head amino acid (HAA) composed of any amino acid whether natural or synthetic, the SG is a spacer amino acid (SAA) composed of any amino acid whether natural or synthetic and the AG is an attachment amino acid (AAA) composed of any amino acid whether natural or synthetic. The preferred arrangement may be shown by the following schematic: HAA-SAA-AAA-SAA-AAA-SAA-AAA-SAA-AAA. Other embodiments may be shown more generally by the following schematics:
HAA_{X}-SAA_{Y}-AAA_{A}; and
HAA_{X}[-SAA_{Y}-AAA_{A}]_{Z};
wherein the arrangement of HAA, SG and AG may be in a linear or branched chain arrangement; and wherein there are always the appropriate number of bonds between chemical groups; and wherein X represents the number of HAA groups arranged in succession or any other arrangement such as a branched chain in the head region of the linker, and wherein Y represents the number of spacer groups arranged in succession or any other arrangement such as a branched chain in the body region of the linker; and wherein A represents the number of attachment groups 112 arranged in succession or any other arrangement such as branched chain in the body region 110 of the linker 120, and wherein Z represents the number of repeating -SAA_{Y}-AAA_{A} groups arranged in succession or any other arrangement such as a branched chain in the body region of the linker, and wherein X ranges from 1 to 20, or from 2 to 8, or from 2 to 6 or from 2 to 4 and Y ranges from 0 to 50, or from 0 to 40 or from 0 to 30 or from 0 to 20 or from 0 to 10 or from 0 to 5 and A ranges from 1 to 50, or from 1 to 40, or from 1 to 30, or from 1 to 20, or from 1 to 10, or from 1 to 5; and Z may range from 1 to 10, or from 2 to 8, or from 2 to 6, or from 2 to 4.

Examples of amino acids that may be used in the tunable peptide linker attachment site include: aspartic acid, glutamic acid, any natural or custom synthesized amino acid having an extra functional group, e.g., lysine having an extra amino group, and mixtures of any of the foregoing. The arrangement may be shown by the following schematics:
HAA_{X}-SAA_{Y}-Asp_{A};
HAA_{X}-SAA_{Y}-Glu_{A};
HAA_{X}-SAA_{Y}-Lys_{A};
HAA_{X}[-SAA_{Y}-Asp_{A}]_{Z};
HAA_{X}[-SAA_{Y}-Glu_{A}]_{Z}; and
HAA_{X}[-SAA_{Y}-Lys_{A}]_{Z};
wherein A represents the number of Asp, Gly or Lys groups in the linker attachment site; and wherein Z represents the number of repeating -SAA_{Y}-Asp_{A} groups in the linker attachment site. In an embodiment the number of aspartic acids in the linker body ranges from 1 to 20 or from 1 to 10 or from 1 to 5. In a preferred embodiment, the number of aspartic acids at the linker body is 1. In an embodiment the number of glutamic acids in the linker body ranges from 1 to 20 or from 1 to 10 or from 1 to 5. In a preferred embodiment, the number of glutamic acids at the linker body is 1. In an embodiment the number of lysines in the linker body ranges from 1 to 20 or from 1 to 10 or from 1 to 5. In a preferred embodiment, the number of lysines at the linker body is 1. In a preferred embodiment, the amino acid at the attachment site (AAA) is glutamic acid.

In some embodiments, selecting Glu or Asp as the AAA makes the linker very hydrophilic. The current state of the art uses hydrophobic chemical chains to bind to ligands. Hydrophobic chemical chains undergo non-specific binding (NSB) with errant molecules in the analyte solution which causes instrument noise. Selecting a hydrophilic AAA in the linker such as glutamic and aspartic acids reduces instrument noise below current levels. Reducing instrument noise improves signal to noise ratio in the analysis.

In some embodiments, the HG, SG and AG are saccharides. In another embodiment, the HG is a head saccharide (HS) composed of any saccharide whether natural or synthetic, the SG is a spacer saccharide (SS) composed of any saccharide whether natural or synthetic and the AG is an attachment saccharide (AS) composed of any saccharide whether natural or synthetic. The preferred arrangement may be shown by the following schematic: HS-SS-AS-SS-AS-SS-AS-SS-AS. Other embodiments may be shown more generally by the following schematics:
HS_{X}-SS_{Y}-AS_{A}; and
HS_{X} [-SS_{Y}-AS_{A}]_{Z};
wherein the arrangement of HS, SS and AS may be in a linear or branched chain arrangement; and wherein there are the appropriate number of bonds between chemical groups; and wherein X represents the number of HS groups arranged in succession or any other arrangement such as a branched chain in the head region of the linker; and wherein Y represents the number of spacer groups arranged in succession or any other arrangement such as a branched chain in the body region of the linker; and wherein A represents the number of attachment groups 112 arranged in succession or any other arrangement such as branched chain in the body region 110 of the linker 120; and wherein Z represents the number of repeating -SS_{Y}-AS_{A} groups arranged in succession or any other arrangement such as a branched chain in the body region of the linker, and wherein X ranges from 1 to 20, or from 2 to 8, or from 2 to 6 or from 2 to 4 and Y ranges from 0 to 50, or from 0 to 40 or from 0 to 30 or from 0 to 20 or from 0 to 10 or from 0 to 5 and A ranges from 1 to 50, or from 1 to 40, or from 1 to 30, or from 1 to 20, or from 1 to 10, or from 1 to 5; and Z may range from 1 to 10, or from 2 to 8, or from 2 to 6, or from 2 to 4.

Examples of saccharides that may be used in the tunable linker attachment site include: dextrose, amylose, galactose, any natural or custom synthesized saccharide having an extra functional group (e.g., dextrose having a thiol group) and mixtures of any of the foregoing. The arrangement may be shown by the following schematics:
HS_{X}-SS_{Y}-AS_{A};
wherein A represents the number of carboxylate or amine groups in the linker attachment site; and wherein Z represents the number of repeating -SS_{Y}-AS_{A} groups in the linker attachment site. In an embodiment the number of carboxylate or amine groups in the linker body ranges from 1 to 20 or from 1 to 10 or from 1 to 5. In a preferred embodiment, the number of aspartic acids at the linker body is 1. In an embodiment, the number of carboxylate groups in the linker body ranges from 1 to 20 or from 1 to 10 or from 1 to 5. In a preferred embodiment, the number of carboxylate groups at the linker body is 1. In an embodiment the number of amine groups in the linker body ranges from 1 to 20 or from 1 to 10 or from 1 to 5. In a preferred embodiment, the number of amine groups at the linker body is 1. In a preferred embodiment, the saccharide at the attachment site (AS) is dextrose. In some embodiments, the saccharide at the AS is substituted with R′, —C(O)—R′, or —C(O)NR′, wherein R′ is hydrogen, C₁-C₁₀ alkyl, C₁-C₁₀ alkenyl, C₁-C₁₀ alkynyl, C₁-C₁₀ carbocycle, C₁-C₁₀ heterocycle, C₁-C₁₀ aryl, C₁-C₁₀ heteroaryl, or a combination thereof. Each of HS, SS, or AS may be connected via a 1.2-, 1.3-, 1.4-, 1,5-, or 1.6 glycosidic linkage, or a combination thereof.

In some embodiments, selecting Glu or Asp as the AAA makes the linker very hydrophilic. The current state of the art uses hydrophobic chemical chains to bind to ligands. Hydrophobic chemical chains undergo non-specific binding (NSB) with errant molecules in the analyte solution which causes instrument noise. Selecting a hydrophilic AAA in the linker such as glutamic and aspartic acids reduces instrument noise below current levels. Reducing instrument noise improves signal to noise ratio in the analysis.

In some embodiments, the one or more attachment amino acid (AAA) or attachment saccharide (AS) sites located in the linker body may contain any chemical functional group which may be used to couple with the ligand. In some embodiments of the disclosure, carboxylic acid functional groups (e.g., —COOH) may be used as attachment sites. In some embodiments the carboxylic acid functional groups may derive from a nitrilotriacetic acid (NTA) functional group. In some embodiments, the AAA or AS sites may contain amino groups, such as those derived from tris-(2-aminoethyl)amine. Although it will be appreciated that a wide variety of chemical reactions are possible within the scope of the disclosure, the carboxylic acid functional groups may undergo dehydration, as one example, with functional groups on the ligands to form a bond. In another embodiment the attachment site may be an amino functional group. In one example, the amino functional groups may undergo dehydration, as one example, with functional groups on the ligands to form a bond. In another embodiment, the attachment site may be a biotin molecule. In another embodiment, the attachment site may be one or more nitrilotriacetic acid (NTA) molecules. In another embodiment the attachment site may be any functional group which may be chemically converted to carboxylic acid functional groups. In another embodiment the attachment site may be any functional group which may be chemically converted to amino functional groups. In another embodiment the attachment site may be any functional group which may be chemically converted to a nitrilotriacetic acid (NTA) molecule. In another embodiment, the attachment site may come from any natural or synthetic amino acid, saccharide, or other moiety with a functional group which may bind to a ligand on its own or may be chemically modified to bind to a ligand.

In some embodiments, the attachment sites may further comprise a cation. In some embodiments, the cation is a metal ion. The attachment sites 112, including carboxylates or other chemical groups, may chelate to the metal ion. In some instances, the metal ion comprises a transition metal ion. In some embodiments, the transition metal ion comprises nickel, iron, cobalt, copper, or zinc. In some embodiments, the metal ion is selected from the group of nickel, iron, cobalt, copper, zinc, calcium, sodium, magnesium, and a combination thereof. In some embodiments, the metal ion chelates to the attachment sites 112 and the ligand 114.

The number of attachment sites (e.g., carboxylic acids or NTAs) and spacer (e.g., NPC) units may be selected to achieve a desired density of attachment sites on the linker as shown in FIG. 3. For example, larger ligands may require lower attachment site density 304, while smaller ligands may support a larger attachment site density 302. Similarly, larger analytes may require lower ligand density, while smaller analytes may support a larger ligand density.

In some embodiments, the linker may be a matrix, and the matrix may comprise a matrix thickness. In some embodiments, the matrix thickness may range from about 1 nm to about 100 nm. In some embodiments, matrix thickness may range from about 1 nm to about 100 nm, from about 10 nm to about 90 nm, from about 20 nm to about 80 nm, from about 30 nm to about 70 nm, or from about 40 nm to about 60 nm. In some embodiments, the matrix thickness is at least 10 nm. In some embodiments, the matrix thickness is at most 100 nm. In some embodiments, the matrix thickness is no more than 1000 nm. In some embodiments, the matrix thickness is no less than 1 nm.

In some cases, fewer PAG or spacer groups, represented by wavy lines 306 in FIG. 3, and more amino acids (with —COOH functional groups for example) or one or more NTA molecules may be used to increase the density of the attachment sites and therefore increase the density of ligands able to bind to the linker. In another embodiment, more PAG groups 308 can be added with fewer amino acids or fewer NTA molecules in the linker which creates a larger distance between attachment sites and thereby decreases the density of ligands able to bind to the linker. In an embodiment of the disclosure, the density of the linker attachment sites is tunable.

Increasing the number of immobilized ligands (e.g., proteins) bound to the linker increases the number of analyte molecules captured which directly increases SPR and LSPR signal strength. However, one cannot solely increase the number of the attachment (e.g., —COOH which may derive from NTA molecules) functional groups on the tunable linker with the expectation of a linear increase in analyte capture. The size and shape of the immobilized ligand (e.g., protein) and the analyte must also be considered. The linker-ligand may be tuned to increase analyte capture given steric constraints of the ligands and analytes. In some embodiments where the ligand (e.g., protein) is large, a higher density of attachment (e.g., —COOH which may derive from NTA molecules) sites might sterically impede an increased concentration of immobilized proteins. Therefore, in some embodiments, the novel linker may be tuned with an increased number of PAG units and a decreased number of amino acid or NTA sites 304. This embodiment would increase the length of the linker and decrease the density 304 of attachment sites. In some embodiments such as this example, a decreased number of attachment (e.g., —COOH) sites is preferred. In some embodiments the —COOH sites may derive from one or more NTA molecules.

In other embodiments, where the ligand (e.g., protein) is not as large, a higher density of attachment sites might not have the steric considerations discussed above and in fact it may be desirable to bind a higher concentration of ligands (e.g., proteins). Therefore, in some embodiments, the novel linker may be tuned with a decreased number of PAG units and an increased number of amino acid or NTA attachment sites 302. This embodiment would decrease the length of the spacer between attachment sites and therefore increase the density 302 of attachment (e.g., —COOH) sites. In some embodiments such as this example, an increased number of attachment (e.g., —COOH) sites is preferred. In some embodiments the —COOH sites may derive from one or more NTA molecules.

In some embodiments, the linker body may be a branched PAG without requiring the use of amino acids. The branched PAG may include branches terminated with attachment sites for ligands, such as carboxylic acid sites. In some embodiments, the branched PAG may include branches terminated with attachment sites for ligands such as one or more NTA sites.

Examples of tunable linkers include but are not limited to:
Cys_{X}[-Gly_{Y}-Asp_{A}]_{Z},
Cys_{X}[-Gly_{Y}-Glu_{A}]_{Z},
Cys_{X}[-Gly_{Y}-Lys_{A}]_{Z},
Cys_{X}[-Gly_{Y}-NTA_{A}]_{Z};
Cys_{X}[-PAG_{Y}-Asp_{A}]_{Z};
Cys_{X}[-PAG_{Y}-Glu_{A}]_{Z};
Cys_{X}[-PAG_{Y}-Lys_{A}]_{Z};
Cys_{X}[-PAG_{Y}-NTA_{A}]_{Z};
Cys_{X}[-NPC_{Y}-Asp_{A}]_{Z};
Cys_{X}[-NPC_{Y}-Glu_{A}]_{Z};
Cys_{X}[-NPC_{Y}-Lys_{A}]_{Z};
Cys_{X}[-NPC_{Y}-NTA_{A}]_{Z};
Cys_{X}[-(CH₂)_{Y}-Asp_{A}]_{Z};
Cys_{X}[-(CH₂)_{Y}-Glu_{A}]_{Z};
Cys_{X}[-(CH₂)_{Y}-Lys_{A}]_{Z};
Cys_{X}[-(CH₂)_{Y}-NTA_{A}]_{Z};
Met_{X}[-Gly_{Y}-Asp_{A}]_{Z};
Met_{X}[-Gly_{Y}-Glu_{A}]_{Z};
Met_{X}[-Gly_{Y}-Lys_{A}]_{Z};
Met_{X}[-Gly_{Y}-NTA_{A}]_{Z};
Met_{X}[-PAG_{Y}-Asp_{A}]_{Z};
Met_{X}[-PAG_{Y}-Glu_{A}]_{Z};
Met_{X}[-PAG_{Y}-Lys_{A}]_{Z};
Met_{X}[-PAG_{Y}-NTA_{A}]_{Z};
Met_{X}[-NPC_{Y}-Asp_{A}]_{Z};
Met_{X}[-NPC_{Y}-Glu_{A}]_{Z};
Met_{X}[-NPC_{Y}-Lys_{A}]_{Z};
Met_{X}[-NPC_{Y}-NTA_{A}]_{Z};
Met_{X}[-(CH₂)_{Y}-Asp_{A}]_{Z};
Met_{X}[-(CH₂)_{Y}-Glu_{A}]_{Z};
Met_{X}[-(CH₂)_{Y}-Lys_{A}]_{Z};
Met_{X}[-(CH₂)_{Y}-NTA_{A}]_{Z},
Da_{X}[-Gly_{Y}-Asp_{A}]_{Z};
Da_{X}[-Gly_{Y}-Glu_{A}]_{Z},
Da_{X}[-Gly_{Y}-Lys_{A}]_{Z},
Da_{X}[-Gly_{Y}-NTA_{A}]_{Z};
Da_{X}[-PAG_{Y}-Asp_{A}]_{Z};
Da_{X}[-PAG_{Y}-Glu_{A}]_{Z},
Da_{X}[-PAG_{Y}-Lys_{A}]_{Z},
Da_{X}[-PAG_{Y}-NTA_{A}]_{Z};
Da_{X}[-NPC_{Y}-Asp_{A}]_{Z},
Da_{X}[-NPC_{Y}-Lys_{A}]_{Z},
Da_{X}[-NPC_{Y}-NTA_{A}]_{Z};
Da_{X}[-(CH₂)_{Y}-Asp_{A}]_{Z};
Da_{X}[-(CH₂)_{Y}-Glu_{A}]_{Z};
Da_{X}[-(CH₂)_{Y}-Lys_{A}]_{Z};
Da_{X}[-(CH₂)_{Y}-NTA_{A}]_{Z};
SR_{X}[-Gly_{Y}-Asp_{A}]_{Z},
SR_{X}[-Gly_{Y}-Glu_{A}]_{Z},
SR_{X}[-Gly_{Y}-Lys_{A}]_{Z},
SR_{X}[-Gly_{Y}-NTA_{A}]_{Z};
SR_{X}[-PAG_{Y}-Asp_{A}]_{Z};
SR_{X}[-PAG_{Y}-Glu_{A}]_{Z};
SR_{X}[-PAG_{Y}-Lys_{A}]_{Z};
SR_{X}[-PAG_{Y}-NTA_{A}]_{Z};
SR_{X}[-NPC_{Y}-Asp_{A}]_{Z};
SR_{X}[-NPC_{Y}-Glu_{A}]_{Z};
SR_{X}[-NPC_{Y}-Lys_{A}]_{Z};
SR_{X}[-NPC_{Y}-NTA_{A}]_{Z};
SR_{X}[-(CH₂)_{Y}-Asp_{A}]_{Z};
SR_{X}[-(CH₂)_{Y}-Glu_{A}]_{Z};
SR_{X}[-(CH₂)_{Y}-Lys_{A}]_{Z};
NR_{X}[-Gly_{Y}-Asp_{A}]_{Z};
NR_{X}[-Gly_{Y}-Glu_{A}]_{Z},
NR_{X}[-Gly_{Y}-Lys_{A}]_{Z},
NR_{X}[-Gly_{Y}-NTA_{A}]_{Z};
NR_{X}[-PAG_{Y}-Asp_{A}]_{Z};
NR_{X}[-PAG_{Y}-Glu_{A}]_{Z};
NR_{X}[-PAG_{Y}-Lys_{A}]_{Z};
NR_{X}[-PAG_{Y}-NTA_{A}]_{Z};
NR_{X}[-NPC_{Y}-Asp_{A}]_{Z};
NR_{X}[-NPC_{Y}-Glu_{A}]_{Z};
NR_{X}[-NPC_{Y}-Lys_{A}]_{Z};
NR_{X}[-NPC_{Y}-NTA_{A}]_{Z};
NR_{X}[-(CH₂)_{Y}-Asp_{A}]_{Z};
NR_{X}[-(CH₂)_{Y}-Glu_{A}]_{Z};
NR_{X}[-(CH₂)_{Y}-Lys_{A}]_{Z};
NR_{X}[-(CH₂)_{Y}-NTA_{A}]_{Z};
Cys_{X}-Gly_{Y}-Asp_{A};
Cys_{X}-Gly_{Y}-Glu_{A};
Cys_{X}-Gly_{Y}-Lys_{A};
Cys_{X}-Gly_{Y}-NTA_{A};
Cys_{X}-PAG_{Y}-Asp_{A};
Cys_{X}-PAG_{Y}-Glu_{A};
Cys_{X}-PAG_{Y}-Lys_{A};
Cys_{X}-PAG_{Y}-NTA_{A};
Cys_{X} NPC_{Y}-Asp_{A};
Cys_{X} NPC_{Y}-Glu_{A};
Cys_{X}-NPC_{Y}-Lys_{A};
Cys_{X}-NPC_{Y}-NTA_{A};
Cys_{X}-(CH₂)_{Y}-Asp_{A};
Cys_{X}-(CH₂)_{Y}-Glu_{A};
Cys_{X}-(CH₂)_{Y}-Lys_{A};
Cys_{X}-(CH₂)_{Y}-NTA_{A};
Met_{X}-Gly_{Y}-Asp_{A};
Met_{X}-Gly_{Y}-Glu_{A};
Met_{X}-Gly_{Y}-Lys_{A};
Met_{X}-Gly_{Y}-NTA_{A};
Met_{X}-PAG_{Y}-Asp_{A};
Met_{X}-PAG_{Y}-Glu_{A};
Met_{X}-PAG_{Y}-Lys_{A};
Met_{X}-PAG_{Y}-NTA_{A};
Met_{X}-NPC_{Y}-Asp_{A};
Met_{X}-NPC_{Y}-Glu_{A};
Met_{Z}-NPC_{Y}-Lys_{A};
Met_{X}-NPC_{Y}-NTA_{A};
Met_{X}-(CH₂)_{Y}-Asp_{A};
Met_{X}-(CH₂)_{Y}-Glu_{A};
Met_{X}-(CH₂)_{Y}-Lys_{A};
Met_{X}-(CH₂)_{Y}-NTA_{A};
Da_{X}-Gly_{Y}-Asp_{A};
Da_{X}-Gly_{Y}-Glu_{A};
Da_{X}-Gly_{Y}-Lys_{A};
Da_{X}-Gly_{Y}-NTA_{A};
Da_{X}-PAG_{Y}-Asp_{A};
Da_{X}-PAG_{Y}-Glu_{A};
Da_{X}-PAG_{Y}-Lys_{A};
Da_{X}-PAG_{Y}-NTA_{A};
Da_{X}-NPC_{Y}-Asp_{A};
Da_{X}-NPC_{Y}-Glu_{A};
Da_{X}-NPC_{Y}-Lys_{A};
Da_{X}-NPC_{Y}-NTA_{A};
Da_{X}-(CH₂)_{Y}-Asp_{A};
Da_{X}-(CH₂)_{Y}-Glu_{A};
Da_{X}-(CH₂)_{Y}-Lys_{A};
Da_{X}-(CH₂)_{Y}-NTA_{A};
SR_{X}-Gly_{Y}-Asp_{A};
SR_{X}-Gly_{Y}-Glu_{A};
SR_{X}-Gly_{Y}-Lys_{A};
SR_{X}-Gly_{Y}-NTA_{A};
SR_{X}-PAG_{Y}-Asp_{A};
SR_{X}-PAG_{Y}-Glu_{A};
SR_{X}-PAG_{Y}-Lys_{A};
SR_{X}-PAG_{Y}-NTA_{A};
SR_{X}-NPC_{Y}-Asp_{A};
SR_{X}-NPC_{Y}-Glu_{A};
SR_{X}-NPC_{Y}-Lys_{A};
SR_{X}-NPC_{Y}-NTA_{A};
SR_{X}-(CH₂)_{Y}-Asp_{A};
SR_{X}-(CH₂)_{Y}-Glu_{A};
SR_{X}-(CH₂)_{Y}-Lys_{A};
SR_{X}-(CH₂)_{Y}-NTA_{A};
NR_{X}-Gly_{Y}-Asp_{A};
NR_{X}-Gly_{Y}-Glu_{A};
NR_{X}-Gly_{Y}-Lys_{A};
NR_{X}-Gly_{Y}-NTA_{A};
NR_{X}-PAG_{Y}-Asp_{A};
NR_{X}-PAG_{Y}-Glu_{A};
NR_{X}-PAG_{Y}-Lys_{A};
NR_{X}-PAG_{Y}-NTA_{A};
NR_{X}-NPC_{Y}-Asp_{A};
NR_{X}-NPC_{Y}-Glu_{A};
NR_{X}-NPC_{Y}-Lys_{A};
NR_{X}-NPC_{Y}-NTA_{A};
NR_{X}-(CH₂)_{Y}-Asp_{A};
NR_{X}-(CH₂)_{Y}-Glu_{A};
NR_{X}-(CH₂)_{Y}-Lys_{A}; and
NR_{X}-(CH₂)_{Y}-NTA_{A};
wherein X ranges from 1 to 20, or from 2 to 8, or from 2 to 6 or from 2 to 4 and Y ranges from 0 to 50, or from 0 to 40 or from 0 to 30 or from 0 to 20 or from 0 to 10 or from 0 to 5; and A ranges from 1 to 50, or from 1 to 40 or from 1 to 30 or from 1 to 20 or from 1 to 10 or from 1 to 5; and Z may range from 1 to 10, or from 2 to 8, or from 2 to 6, or from 2 to 4.

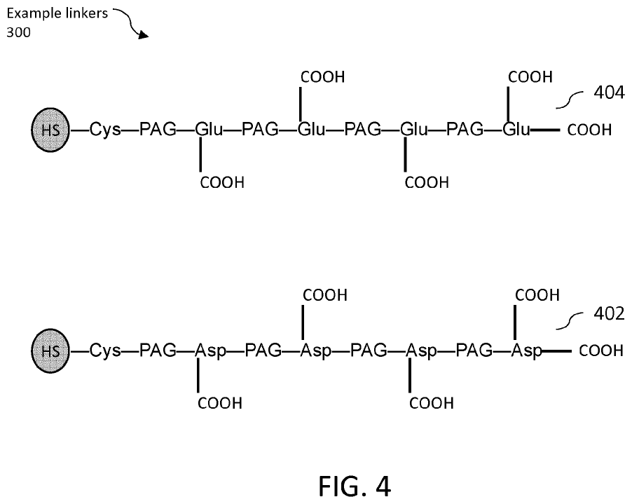

FIG. 4 illustrates exemplary SPR or LSPR tunable linker molecules wherein the head group may be a cysteine (Cys), the spacer molecule may be a PAG for example and the attachment amino acid may be a glutamic acid or an aspartic acid with an example of attachment sites being a —COOH functional group. FIG. 4 shows an example of Cys_{X}[-PAG_{Y}-Asp_{A}]_{Z} 402 and Cys_{X}[-PAG_{Y}-Glu_{A}]_{Z} 404, wherein X is 1, wherein the number of PAG units, Y, can vary as described above and Z, the number of repeating -PAG-Glu and -PAG-Asp units is 4.

For the embodiments disclosed herein, the following generic formula may also apply:
HG_{X}-SG_{Y}-AG_{A};
wherein the arrangement of HG, SG and AG may be in a linear or branched chain arrangement; and wherein there are the appropriate number of bonds between chemical groups; and wherein X represents the number of HG groups arranged in succession or any other arrangement such as a branched chain in the head region of the linker, and wherein Y represents the number of spacer groups arranged in succession or any other arrangement such as a branched chain in the body region of the linker, and wherein A represents the number of AG groups 112 arranged in succession (e.g., linear arrangement) or any other arrangement such as a branched chain in the body region 110 of the linker 120, and wherein X ranges from 1 to 20, or from 2 to 8, or from 2 to 6 or from 2 to 4 and Y ranges from 0 to 50, or from 0 to 40 or from 0 to 30 or from 0 to 20 or from 0 to 10 or from 0 to 5 and A may range from 1 to 10, or from 1 to 8, or from 1 to 6, or from 1 to 4.

Formulas I, II, III, and IV (below) show additional exemplary linker structures.

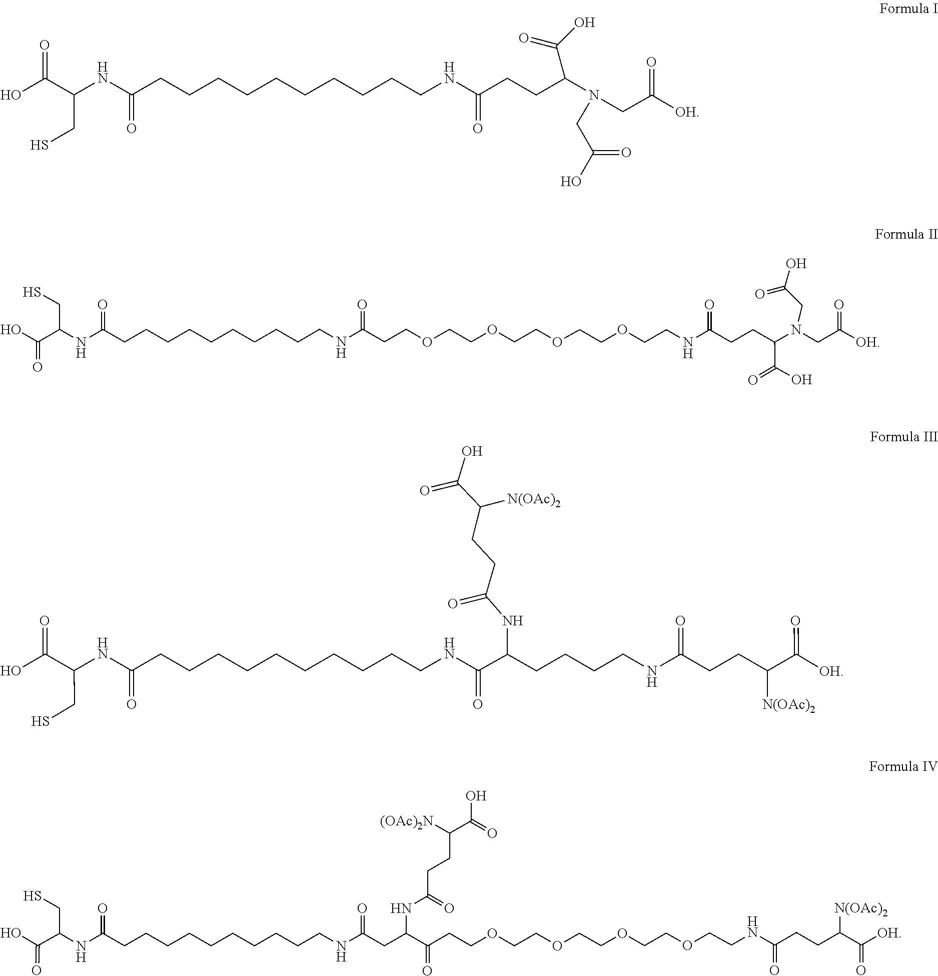

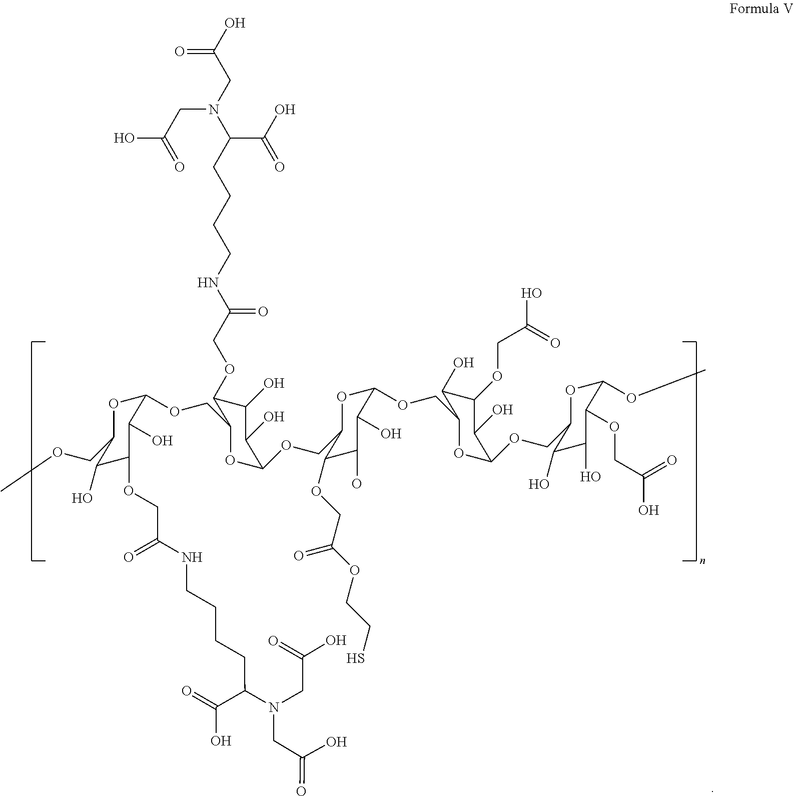

These examples show further embodiments of the present disclosure wherein the synthesized linker may further consider not only steric requirements of the ligand, density of the attachment sites but also tunable hydrophobicity and hydrophilicity of the linker, spacer groups and attachment groups. In these example structures, a cysteine amino acid for example may provide the source of the thiol functional group used for anchoring to a metal (e.g., gold) surface.

In some embodiments, the linker may be designed with the following characteristics such as the following schematics, wherein HG is one or more header groups, LM is a lipophilic moiety, SG is a spacer group and HM is a hydrophilic moiety:
HG_{X}-LM_{L}-SG_{Y}-HM_{H};
HG_{X}-HM_{H}-SG_{Y}-LM_{L};
HG_{X}-SG_{Y}-LM_{L}-HM_{H};
HG_{X}-SG_{Y}-HM_{H}-LM_{L};
HG_{X}-[LM_{L}-SG_{Y}-HM_{H}]_{Z};
HG_{X}-[LM_{L}-SG_{Y}]_{Z}-HM_{H}; and
HG_{X}-LM_{L}-[SG_{Y}-HM_{H}]_{Z};
wherein the arrangement of HG, LM, spacer groups and hydrophilic groups may be in a linear or branched chain arrangement; and wherein there are the appropriate number of bonds between chemical groups; and wherein X represents the number of head 108 groups arranged in succession or any other arrangement such as branched chain in the head 108 region of the linker 120, and wherein L represents the number of lipophilic 110 groups arranged in succession or any other arrangement such as branched chain, in the body 110 region of the linker 120, and wherein Y represents the number of spacer 110 groups arranged in succession or any other arrangement such as branched chain in the body 110 region of the linker 120, and where H represents the number of hydrophilic 110 groups arranged in succession or any other arrangement such as branched chain, in the body 110 region of the linker 120, and wherein Z represents the number of repeating groups within the brackets arranged in succession or any other arrangement such as branched chain in the body 110 region of the linker 120, and wherein X ranges from 1 to 20, or from 2 to 8, or from 2 to 6 or from 2 to 4; and L ranges from 0 to 25, or from 1 to 22 or from 1 to 20 or from 1 to 15 or from 1 to 10 or from 1 to 5; and Y ranges from 0 to 50, or from 0 to 40 or from 0 to 30 or from 0 to 20 or from 0 to 10 or from 1 to 5; and H ranges from 0 to 25, or from 1 to 22 or from 1 to 20 or from 1 to 15 or from 1 to 10 or from 1 to 5; and Z may range from 1 to 10, or from 2 to 8, or from 2 to 6, or from 2 to 4.

In some embodiments the linker may include hydrophobic (e.g., lipophilic) spacer groups such as a tunable number of —(CH₂)— groups. This arrangement may be represented by the following schematics:
HG_{X}-(CH₂)_{L}-AG_{A};
HG_{X}[-(CH₂)_{L}-AG_{A}]_{Z};
HG_{X}-(CH₂)_{L}-SG_{Y}-AG_{A};
HG_{X}[-(CH₂)_{L}-SG_{Y}-AG_{A}]_{Z};
HG_{X}-SG_{Y}-(CH₂)_{L}-AG_{A}; and
HG_{X}[-SG_{Y}-(CH₂)_{L}-AG_{A}]_{Z};
wherein the arrangement of HG, SG, CH₂ groups and AG groups may be in a linear or branched chain arrangement; and wherein there are the appropriate number of bonds between chemical groups; and wherein X represents the number of head 108 groups arranged in succession or any other arrangement such as branched chain in the head 108 region of the linker 120, and wherein L represents the number of —(CH₂)— spacer 110 groups arranged in succession or any other arrangement such as branched chain, in the body 110 region of the linker 120, and wherein A represents the number of attachment groups 112 arranged in succession or any other arrangement such as branched chain in the body region 110 of the linker 120, and wherein Y represents the number of spacer groups 110 arranged in succession or any other arrangement such as branched chain in the body region 110 of the linker 120, and wherein Z represents the number of repeating groups within the brackets arranged in succession or any other arrangement such as branched chain in the body 110 region of the linker 120; and wherein X ranges from 1 to 20, or from 2 to 8, or from 2 to 6 or from 2 to 4 and L ranges from 0 to 25, or from 1 to 22 or from 1 to 20 or from 1 to 15 or from 1 to 10 or from 1 to 5 and A ranges from 1 to 50, or from 1 to 40 or from 1 to 30 or from 1 to 20 or from 1 to 10 or from 1 to 5 and Y ranges from 0 to 50, or from 0 to 40, or from 0 to 30, or from 0 to 20, or from 0 to 10 or from 0 to 5; and Z may range from 1 to 10, or from 2 to 8, or from 2 to 6, or from 2 to 4.

In some embodiments, the linker may include hydrophobic (e.g., lipophilic) spacer groups such as a tunable number of —(CH₂)— groups. In some embodiments the linker may include hydrophilic (e.g., lipophobic) spacer groups such as a tunable number of PAG groups. In some embodiments, PAG may be PEG, POE, PPG, PBG, NPC and combinations thereof (e.g., linear or branched polymers including combinations of two or more different PAG subunits, such as two or more different PAG units selected from PEG, POE, PPG, PBG and NPC subunits). In another embodiment, the AG may be a tunable number of NTA functional groups. This arrangement may be represented by the following schematic:
HG_{X}-(CH₂)_{L}-NTA_{A};
HG_{X}[-(CH₂)_{L}-NTA_{A}]_{Z};
HG_{X}-(CH₂)_{L}-PAG_{Y}-NTA_{A};
HG_{X}[-(CH₂)_{L}-PAG_{Y}-NTA_{A}]_{Z};
HG_{X}-PAG_{Y}-(CH₂)_{L}-NTA_{A}; and
HG_{X}[-PAG_{Y}-(CH₂)_{L}-NTA_{A}]_{Z};
wherein the arrangement of HG, PAG, CH₂ groups and AG groups may be in a linear or branched chain arrangement; and wherein there are the appropriate number of bonds between chemical groups; and wherein X represents the number of head 108 groups arranged in succession or any other arrangement such as branched chain in the head 108 region of the linker 120, and wherein L represents the number of —(CH₂)— spacer 110 groups arranged in succession or any other arrangement such as branched chain in the body 110 region of the linker 120, and wherein Y represents the number of PAG spacer 110 groups arranged in succession or any other arrangement such as branched chain in the body 110 region of the linker 120, and wherein Z represents the number of repeating bracketed groups arranged in succession or any other arrangement such as branched chain in the body 110 region of the linker 120, and wherein X ranges from 1 to 20 or from 2 to 8 or from 2 to 6 or from 2 to 4 and L ranges from 0 to 25, or from 1 to 22 or from 1 to 20 or from 1 to 15 or from 1 to 10 or from 1 to 5 and A ranges from 1 to 50 or from 1 to 40 or from 1 to 30 or from 1 to 20 or from 1 to 10 or from 1 to 5 and Y ranges from 0 to 50, or from 0 to 40, or from 0 to 30, or from 0 to 20, or from 0 to 10 or from 0 to 5; and Z may range from 1 to 10 or from 2 to 8 or from 2 to 6, or from 2 to 4.

### EXAMPLES

The following illustrative examples are representative of embodiments of the software applications, systems, and methods described herein and are not meant to be limiting in any way.

### Example 1—Synthesis of a Monofunctional NTA Linker with No Hydrophilicity Modification

Synthesis of a mono functional NTA linker having the structure of Formula I is provided herein.

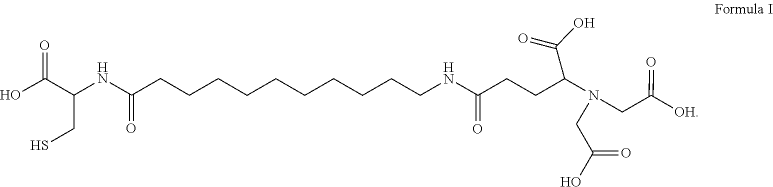 [SMILES: O=C(O)CN(CC(=O)O)C(CCC(=O)NCCCCCCCCCCC(=O)NC(CS)C(=O)O)C(=O)O]

The mono functional NTA linker having the structure of Formula I may be synthesized in the laboratory using standard fmoc chemistry or commercially using the Applied Biosystems 433A peptide synthesizer. Unless otherwise mentioned, chemicals were purchased from Sigma-Aldrich (MilliporeSigma, St. Louis, MO, USA).

As shown in Scheme 1 (below), in a 10 mL solid phase peptide synthesis (SPPS) reactor, 300 mg of 2-Cl-trityl resin 1a was soaked in DCM for 30 minutes. 2-Cl-trityl resin 1a was isolated by vacuum filtration and combined with 176 mg of Fmoc-Cys(Trt)-OH 1b and 210 μL DIPEA in 3 mL of DMF in a peptide shaker (Kamush LP20PR). The mixture was shaken at room temperature (RT) for 60 minutes, and the resulting product 1c was isolated by vacuum filtration and washed three times with DMF and five times with DCM. To cap unreacted functional groups on the resin, 1c was shaken with a 5 mL mixture of DCM/Methanol/DIPEA (85:10:5) for 60 minutes at RT. Subsequently, 1c was isolated by vacuum filtration and washed three times with DMF, six times alternatively with DCM/Methanol, and five times with DCM. To remove the fmoc protecting group, 1c was combined with 3 mL 20% piperidine in DMF with a 30-minute exposure time performed in three steps (10 minutes per exposure). After deprotection, the resin was washed three times with DMF, six times alternatively with DCM/Methanol, and five times with DCM and combined with 509 mg Fmoc-11-aminoundecanoic acid 1d, 558 mg HATU, and 502 μL DIPEA in 3 mL of DMF, and the mixture was shaken at RT for 150 minutes. The resulting product 1e was isolated by vacuum filtration and washed three times with DMF, six times alternatively with DCM/Methanol, and five times with DCM. Reaction completion was tested using the standard Kaiser test (ninhydrin test). To remove the fmoc protecting group, 1e was combined with 3 mL 20% piperidine in DMF with a 30-minute exposure time performed in three steps (10 minutes per exposure). After deprotection, the resin was washed three times with DMF, six times alternatively with DCM/Methanol, and five times with DCM and combined with 523 mg fmoc-L-glutamic acid-OtBu 1f, 553 mg HATU, 502 μL DIPEA in 3 mL of DMF, and the mixture was shaken at RT for 150 minutes. The resulting product 1g was isolated by vacuum filtration and washed three times with DMF, six times alternatively with DCM/Methanol, and five times with DCM. Reaction completion was tested using the standard Kaiser test. To remove the fmoc protecting group the resin was combined with 3 mL 20% piperidine in DMF with a 30-minute exposure time performed in three steps (10 minutes per exposure). After deprotection, the resin was washed three times with DMF, six times alternatively with DCM/Methanol, and five times with DCM and combined with 522 mg IAA and 980 μL DIPEA in 2 mL DMF, and the mixture was shaken at RT overnight. The resulting product 1h was isolated by vacuum filtration and washed three times with DMF, six times alternatively with DCM/Methanol, and five times with DCM. Reaction completion was tested using the standard Kaiser test. 1h was combined with a mixture of TFA/H₂O/TIS (95:2.5:2.5) in the peptide shaker and shaken at RT for 3 hours. The resulting product 1i was precipitated in ether.

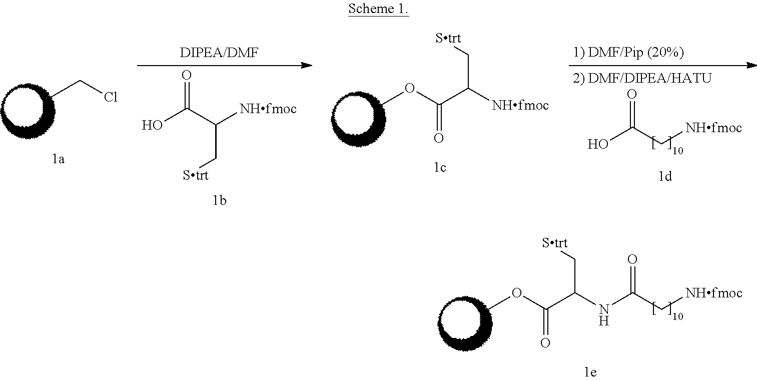

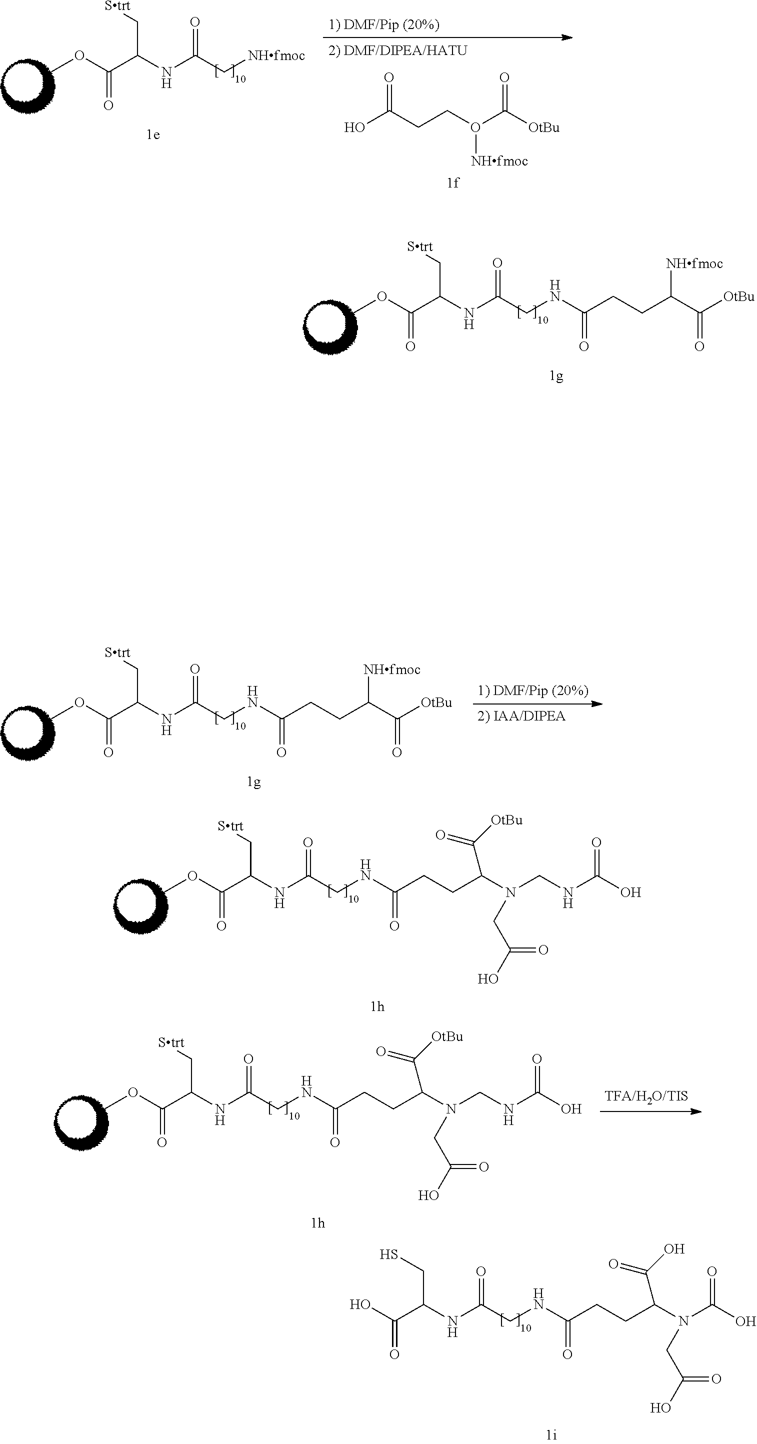

### Example 2—Synthesis of a Monofunctional NTA Linker with a Hydrophilicity Modification

Synthesis of a mono functional NTA linker having the structure of Formula II is provided herein.

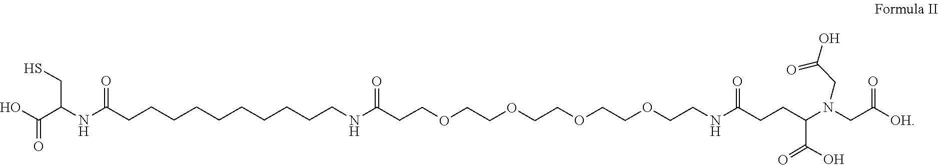 [SMILES: O=C(O)CN(CC(=O)O)C(CCC(=O)NCCOCCOCCOCCOCCC(=O)NCCCCCCCCCCC(=O)NC(CS)C(=O)O)C(=O)O]

The mono functional NTA linker having the structure of Formula II may be synthesized in the laboratory using standard fmoc chemistry or commercially using the Applied Biosystems 433A peptide synthesizer. Unless otherwise mentioned, chemicals were purchased from Sigma-Aldrich (MilliporeSigma, St. Louis, MO, USA).

As shown in Scheme 2 (below), in a 10 mL solid phase peptide synthesis (SPPS) reactor, 300 mg of 2-Cl-trityl resin 2a was soaked in DCM for 30 minutes. 2-Cl-trityl resin 2a was isolated by vacuum filtration and combined with 176 mg of Fmoc-Cys(Trt)-OH 2b and 210 μL DIPEA in 3 mL of DMF in a peptide shaker (Kamush LP20PR). The mixture was shaken at room temperature (RT) for 60 minutes, and the resulting product 2c was isolated by vacuum filtration and washed three times with DMF and five times with DCM. To cap unreacted functional groups on the resin, 2c was shaken with a 5 mL mixture of DCM/Methanol/DIPEA (85:10:5) for 60 minutes at RT. Subsequently, 2c was isolated by vacuum filtration and washed three times with DMF, six times alternatively with DCM/Methanol, and five times with DCM. To remove the fmoc protecting group, 2c was combined with 3 mL 20% piperidine in DMF with a 30-minute exposure time performed in three steps (10 minutes per exposure). After deprotection, the resin was washed three times with DMF, six times alternatively with DCM/Methanol, and five times with DCM and combined with 509 mg Fmoc-11-aminoundecanoic acid 2d, 558 mg HATU, and 502 μL DIPEA in 3 mL of DMF, and the mixture was shaken at RT for 150 minutes. The resulting product 2e was isolated by vacuum filtration and washed three times with DMF, six times alternatively with DCM/Methanol, and five times with DCM. Reaction completion was tested using the standard Kaiser test (ninhydrin test). To remove the fmoc protecting group, 2e was combined with 3 mL 20% piperidine in DMF with a 30-minute exposure time performed in three steps (10 minutes per exposure). After deprotection, the resin was washed three times with DMF, six times alternatively with DCM/Methanol, and five times with DCM and combined with 732 mg fmoc-PEG 2f, 570 mg HATU, and 502 μL DIPEA in 3 mL of DMF, and the mixture was shaken at RT for 150 minutes. The resulting product 2g was isolated by vacuum filtration and was washed three times with DMF, six times alternatively with DCM/Methanol, and five times with DCM. Reaction completion was tested using the standard Kaiser test. To remove the fmoc protecting group, 2g was combined with 3 mL 20% piperidine in DMF with a 30-minute exposure time performed in three steps (10 minutes per exposure). After deprotection, the resin was washed three times with DMF, six times alternatively with DCM/Methanol, and five times with DCM and combined with 523 mg fmoc-L-glutamic acid-OtBu 2h, 553 mg HATU, and 502 μL DIPEA in 3 mL of DMF, and the mixture was shaken at RT for 150 minutes. The resulting product 2i was isolated by vacuum filtration and washed three times with DMF, six times alternatively with DCM/Methanol, and five times with DCM. Reaction completion was tested using the standard Kaiser test. To remove the fmoc protecting group, 2i was combined with 3 mL 20% piperidine in DMF with a 30-minute exposure time performed in three steps (10 minutes per exposure). After deprotection, the resin was washed three times with DMF, six times alternatively with DCM/Methanol, and five times with DCM and combined with 522 mg IAA and 980 μL DIPEA in 2 mL DMF, and the mixture was shaken at RT overnight. The resulting product 2j was isolated by vacuum filtration and washed three times with DMF, six times alternatively with DCM/Methanol, and five times with DCM. Reaction completion was tested using the standard Kaiser test. 2j was combined with a mixture of TFA/H₂O/TIS (95:2.5:2.5) in the peptide shaker and shaken at RT for 3 hours. The resulting product 2k was precipitated in ether.

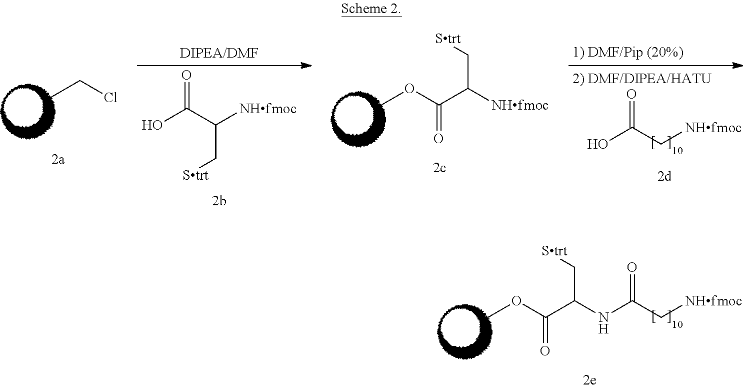

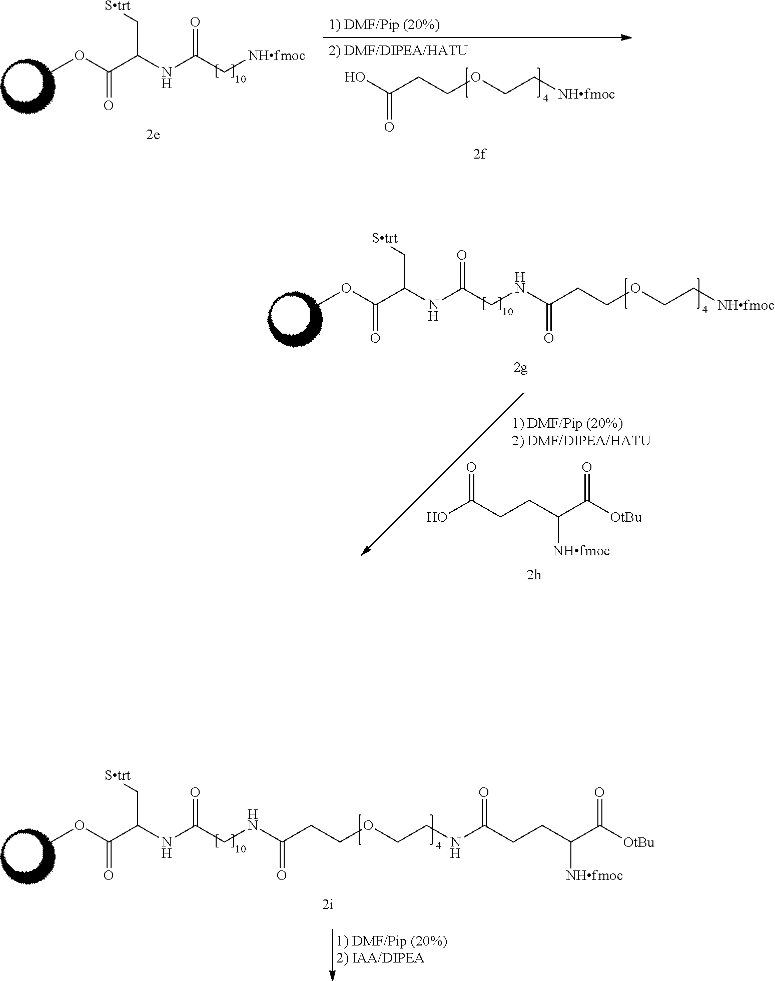

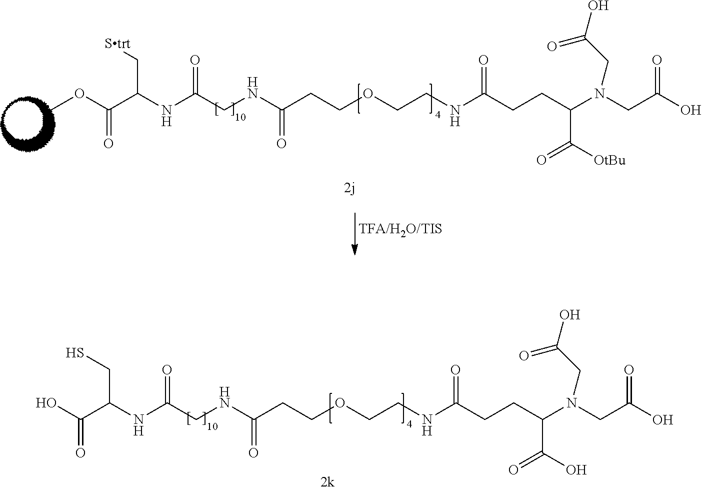

### Example 3—Synthesis of a Bifunctional NTA Linker with No Hydrophilicity Modification

Synthesis of a bifunctional NTA linker having the structure of Formula III is provided herein.

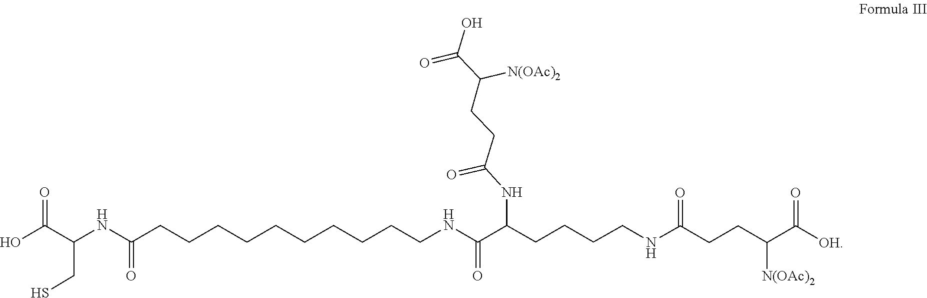

The bifunctional NTA linker having the structure of Formula III may be synthesized in the laboratory using standard fmoc chemistry or commercially using the Applied Biosystems 433A peptide synthesizer. Unless otherwise mentioned, chemicals were purchased from Sigma-Aldrich (MilliporeSigma, St. Louis, MO, USA).

As shown in Scheme 3 (below), in a 10 mL solid phase peptide synthesis (SPPS) reactor, 300 mg of 2-Cl-trityl resin 3a was soaked in DCM for 30 minutes. 2-Cl-trityl resin 3a was isolated by vacuum filtration and combined with 176 mg of Fmoc-Cys(Trt)-OH 3b and 210 μL DIPEA in 3 mL of DMF in a peptide shaker (Kamush LP20PR). The mixture was shaken at room temperature (RT) for 60 minutes, and the resulting product 3c was isolated by vacuum filtration and washed three times with DMF and five times with DCM. To cap unreacted functional groups on the resin, 3c was shaken with a 5 mL mixture of DCM/Methanol/DIPEA (85:10:5) for 60 minutes at RT. Subsequently, 3c was isolated by vacuum filtration and washed three times with DMF, six times alternatively with DCM/Methanol, and five times with DCM. To remove the fmoc protecting group, 3c was combined with 3 mL 20% piperidine in DMF with a 30-minute exposure time performed in three steps (10 minutes per exposure). After deprotection, the resin washed three times with DMF, six times alternatively with DCM/Methanol, and five times with DCM and combined with 509 mg Fmoc-11-aminoundecanoic acid 3d, 558 mg HATU, and 502 μL DIPEA in 3 mL of DMF, and the mixture was shaken at RT for 150 minutes. The resulting product 3e was isolated by vacuum filtration and washed three times with DMF, six times alternatively with DCM/Methanol, and five times with DCM. Reaction completion was tested using the standard Kaiser test (ninhydrin test). To remove the fmoc protecting group, 3e was combined with 3 mL 20% piperidine in DMF with a 30-minute exposure time performed in three steps (10 minutes per exposure). After deprotection, the resin was washed three times with DMF, six times alternatively with DCM/Methanol, and five times with DCM and combined with 887 mg fmoc-Lys (fmoc)-OH 3f, 570 mg HATU, and 502 μL of DIPEA in 3 mL of DMF and shaken at RT for 150 min. The resulting product 3g was isolated by vacuum filtration and washed three times with DMF, six times alternatively with DCM/Methanol, and five times with DCM. Reaction completion was tested using the standard Kaiser test (ninhydrin test). To remove the fmoc protecting group, 3g was combined with 3 mL 20% piperidine in DMF with a 30-minute exposure time performed in three steps (10 minutes per exposure). After deprotection, the resin was washed three times with DMF, six times alternatively with DCM/Methanol, and five times with DCM, and combined with 1,046 mg fmoc-L-glutamic acid-OtBu 3h, 1,106 mg HATU, and 1,004 μL DIPEA in 3 mL of DMF, and the mixture was shaken at RT for 150 minutes. The resulting product 3i was isolated by vacuum filtration and washed three times with DMF, six times alternatively with DCM/Methanol, and five times with DCM. Reaction completion was tested using the standard Kaiser test. To remove the fmoc protecting group, 3i was combined with 3 mL 20% piperidine in DMF with a 30-minute exposure time performed in three steps (10 minutes per exposure). After each exposure, the resin was isolated by vacuum filtration and washed three times with DMF, six times alternatively with DCM/Methanol, and five times with DCM. After deprotection, the resin was combined with 1,044 mg IAA and 1,960 μL DIPEA in 3 mL DMF, and the mixture was shaken at RT overnight. The resulting product 3j was isolated by vacuum filtration and washed three times with DMF, six times alternatively with DCM/Methanol, and five times with DCM. Reaction completion was tested using the standard Kaiser test. 3j was combined with a mixture of TFA/H₂O/TIS (95:2.5:2.5) in the peptide shaker and shaken at RT for 3 hours. The resulting product 3k was precipitated in ether.

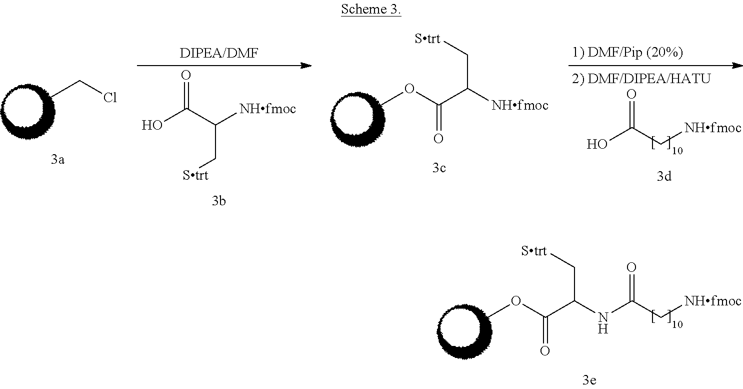

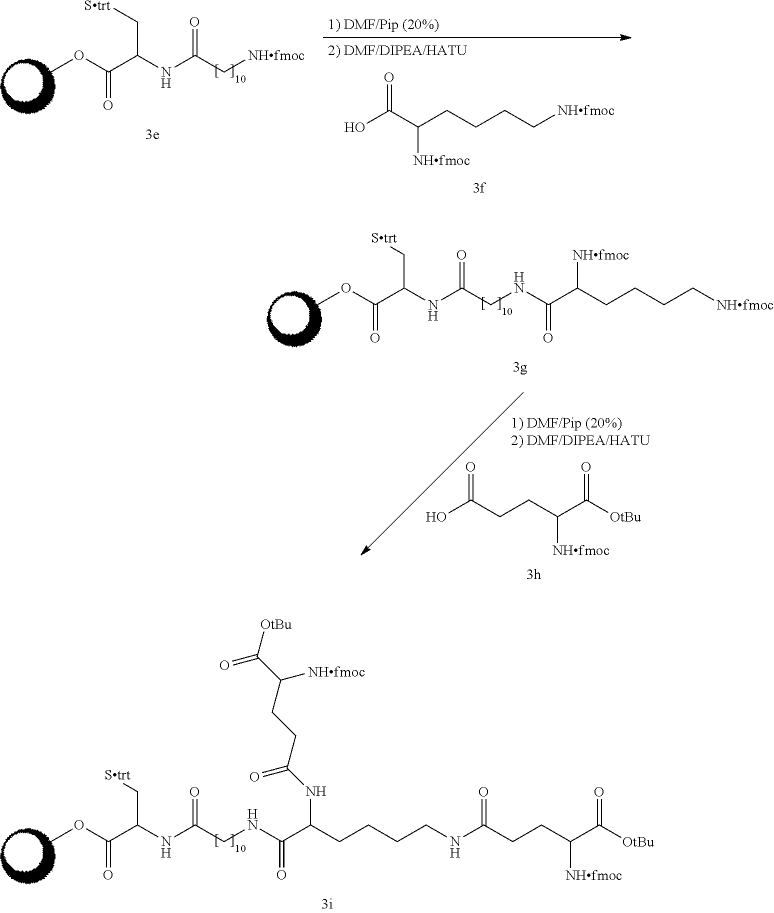

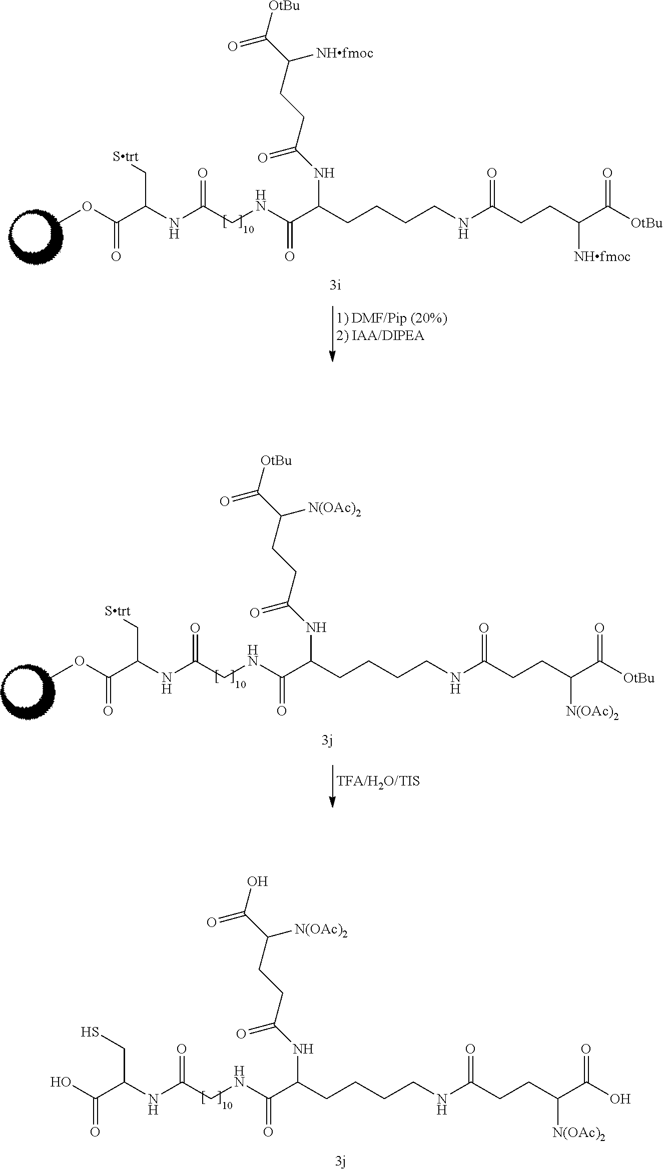

### Example 4—Synthesis of a Bifunctional NTA Linker with a Hydrophilicity Modification

Synthesis of a bifunctional NTA linker having the structure of Formula IV is provided herein.

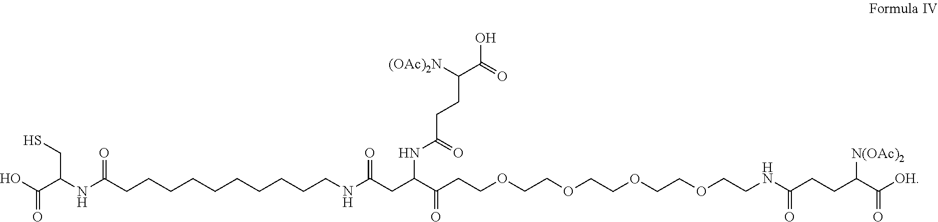

The bifunctional NTA linker having the structure of Formula IV may be synthesized in the laboratory using standard fmoc chemistry or commercially using the Applied Biosystems 433A peptide synthesizer. Unless otherwise mentioned, chemicals were purchased from Sigma-Aldrich (MilliporeSigma, St. Louis, MO, USA).

As shown in Scheme 4 (below), in a 10 mL solid phase peptide synthesis (SPPS) reactor, 300 mg of 2-Cl-trityl resin 4a was soaked in DCM for 30 minutes. 2-Cl-trityl resin 4a was isolated by vacuum filtration and combined with 176 mg of Fmoc-Cys(Trt)-OH 4b and 210 μL DIPEA in 3 mL of DMF in a peptide shaker (Kamush LP20PR). The mixture was shaken at room temperature (RT) for 60 minutes, and the resulting product 4c was isolated by vacuum filtration and washed three times with methanol and five times with DCM. To cap unreacted functional groups on the resin, 4c was shaken with a 5 mL mixture of DCM/Methanol/DIPEA (85:10:5) for 60 minutes at RT. Subsequently, 4c was isolated by vacuum filtration and washed three times with DMF, six times alternatively with DCM/Methanol, and five times with DCM. To remove the fmoc protecting group, 4c was combined with 3 mL 20% piperidine in DMF with a 30-minute exposure time performed in three steps (10 minutes per exposure). After deprotection, the resin was washed three times with DMF, six times alternatively with DCM/Methanol, and five times with DCM and combined with 509 mg Fmoc-11-aminoundecanoic acid 4d, 558 mg HATU, and 502 μL DIPEA in 3 mL of DMF, and the mixture was shaken at RT for 150 minutes. The resulting product 4e was isolated by vacuum filtration and washed three times with DMF, six times alternatively with DCM/Methanol, and five times with DCM. Reaction completion was tested using the standard Kaiser test (ninhydrin test). To remove the fmoc protecting group, de was combined with 3 mL 20% piperidine in DMF with a 30-minute exposure time performed in three steps (10 minutes per exposure). After deprotection, the resin was returned to the peptide shaker and combined with 887 mg Fmoc-D-Lys(ivDde)-OH 4f, 570 mg HATU, and 502 μL of DIPEA in 3 mL of DMF and shaken at RT for 150 min. The resulting product 4g was isolated by vacuum filtration and washed three times with DMF, six times alternatively with DCM/Methanol, and five times with DCM. Reaction completion was tested using the standard Kaiser test (ninhydrin test). To remove the fmoc protecting group, 4g was combined with 3 mL 20% piperidine in DMF with a 30-minute exposure time performed in three steps (10 minutes per exposure). After deprotection, the resin was washed three times with DMF, six times alternatively with DCM/Methanol, and five times with DCM and combined with 732 mg fmoc-PEG 4h. 570 mg HATU, and 502 μL DIPEA in 3 mL of DMF, and the mixture was shaken at RT for 150 minutes. The resulting product 4i was isolated by vacuum filtration and washed three times with DMF, six times alternatively with DCM/Methanol, and five times with DCM. Reaction completion was tested using the standard Kaiser test. To remove the fmoc and iv.Dde protecting groups, 4i was combined with 5 mL of 2% hydrazine in DMF with a 9-minute exposure time performed in three steps (3 minutes per exposure). After each exposure, the resin was isolated by vacuum filtration and washed three times with DMF, six times alternatively with DCM/Methanol, and five times with DCM. After deprotection, the resin and combined 1.046 mg fmoc-L-glutamic acid-OtBu 4j. 1,106 mg HATU, and 1.004 μL DIPEA in 3 mL of DMF, and the mixture was shaken at RT for 150 minutes. The resulting product 4k was isolated by vacuum filtration and washed three times with DMF, six times alternatively with DCM/Methanol, and five times with DCM. Reaction completion was tested using the standard Kaiser test. To remove the fmoc protecting group. 4k was combined with 3 mL 20% piperidine in DMF with a 30-minute exposure time performed in three steps (10 minutes per exposure). After deprotection, the resin was washed three times with DMF, six times alternatively with DCM/Methanol, and five times with DCM and combined with 1.044 mg IAA and 1.960 μL DIPEA in 3 mL DMF, and the mixture was shaken at RT overnight. The resulting product 4l was isolated by vacuum filtration and washed three times with DMF, six times alternatively with DCM/Methanol, and five times with DCM. Reaction completion was tested using the standard Kaiser test. 4l was combined with a mixture of TFA/H₂O/TIS (95:2.5:2.5) in the peptide shaker and shaken at RT for 3 hours. The resulting product 4m was precipitated in ether.

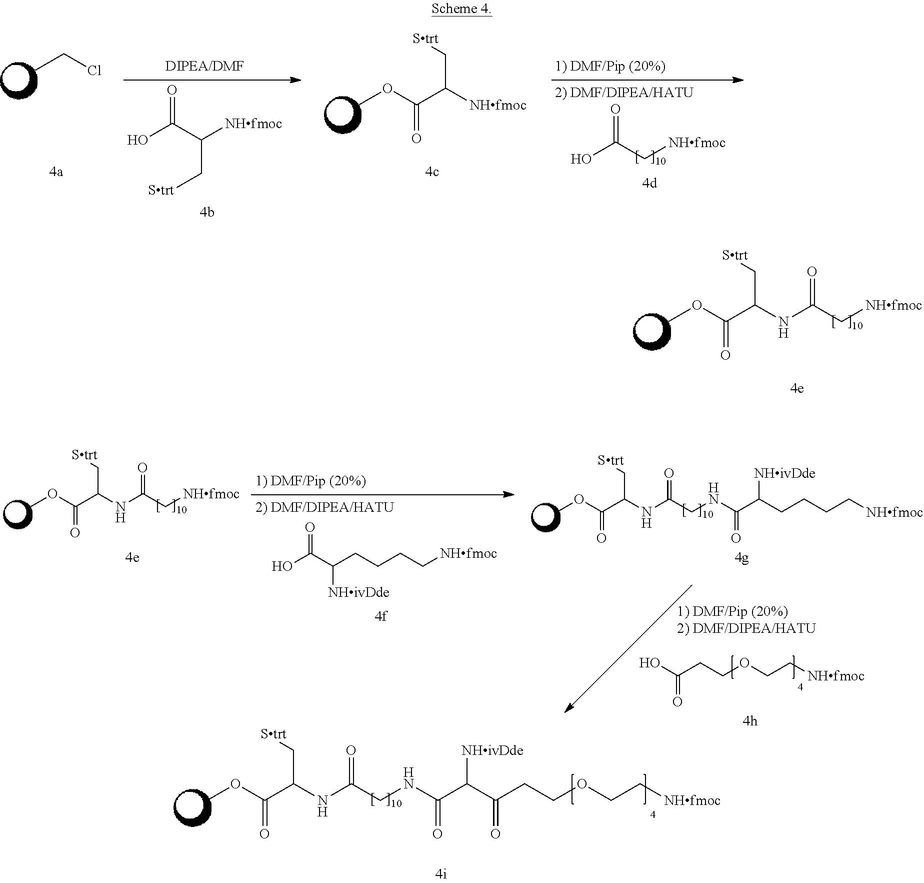

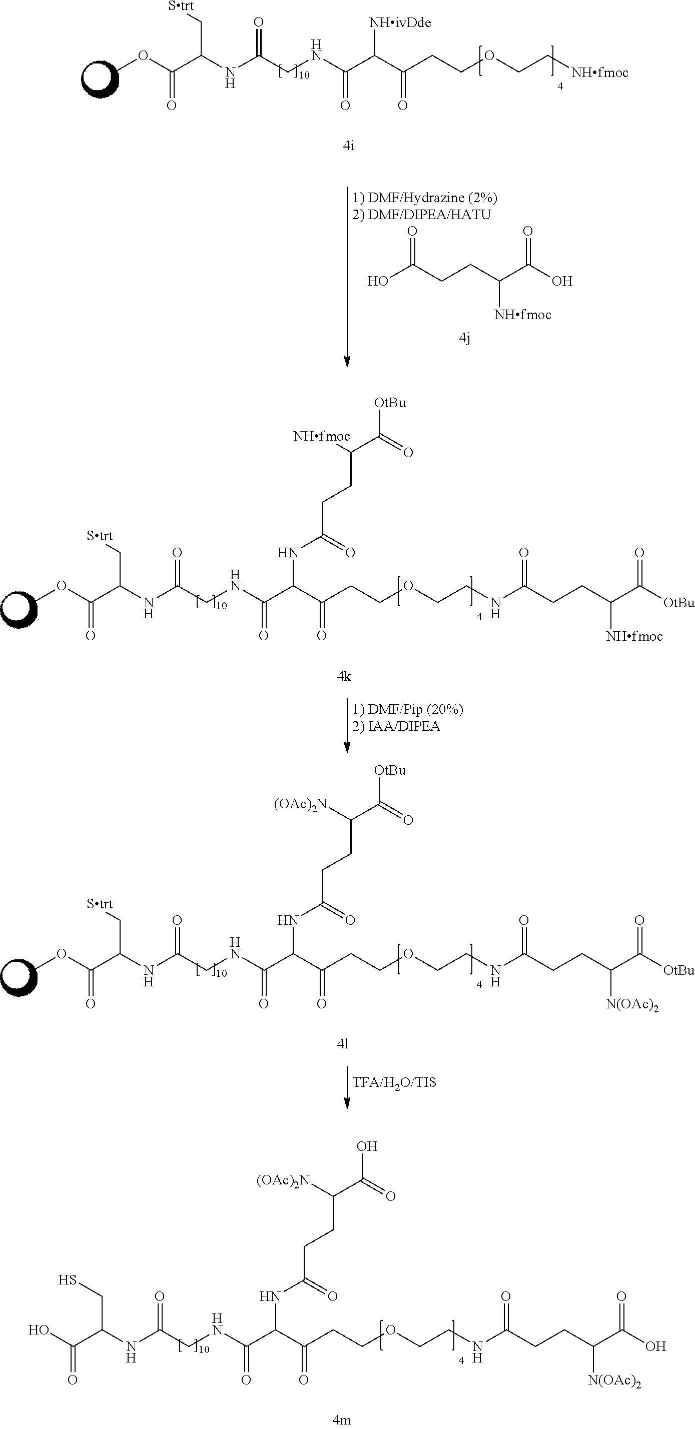

### Example 5—Coating Sensor Surfaces with NTA Linkers

NTA linkers were synthesized as described in Examples 1˜4 (above), and were further disposed over metal (e.g., gold) surfaces to generate a sensor for binding analytes in a sample.

### Glass Chip Sensors

A thiol terminated NTA linker (e.g., formulas I, II, III, or IV above) was dissolved in DI H₂O at a concentration of 1 mM. Bare gold OpenSPR™ sensor chips were cleaned using oxygen plasma for 30 seconds at 40 watts and placed in a petri dish. 100 μL of the NTA solution was pipetted on the surface of each sensor. 1 to 2 mL DI H₂O was added to the petri dish to avoid drying during the incubation. The Petri dish cap was placed and air-tightened using parafilm and incubated overnight. The next day, the sensors were washed using DI H₂O, dried with a nitrogen stream, and stored dry.

### Optical Fiber Sensors

A thiol terminated NTA linker (e.g., formulas I, II, III, or IV above) was dissolved in DI H₂O at a concentration of 1 mM and transferred to the 128-well plate. 128 Alto™ bare gold fibers were placed in 128-fiber racks and cleaned by dipping them in DI H₂O followed by three 5-minute cycles in a sonicator. The fibers were transferred to a drying chamber to dry at RT for 5 minutes. Dried fibers were cleaned using oxygen plasma for 30 seconds at 40 watts, dipped immediately into the NTA aqueous solution, and incubated overnight. The next day, the sensors were washed by dipping the fibers into a 128-well plate containing DI H₂O three times and dried in the drying chamber for 5 minutes. Fabricated NTA sensors were stored dry.

### Example 6—Fabrication of a Sensors with CMD-NTA Linker

Fabrication (i.e., synthesis) of a sensor modified with a CMD-NTA linker having the structure shown below is provided herein.

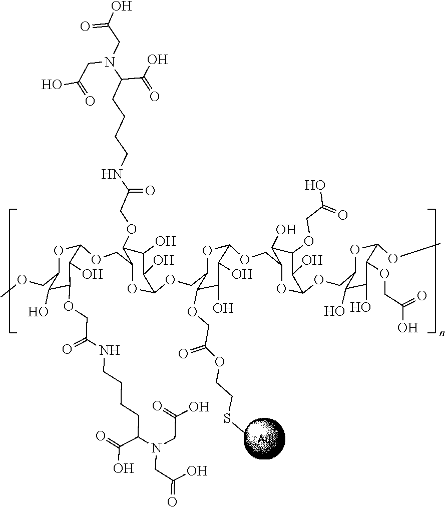

The CMD-NTA modified sensor having the structure of Formula V may be synthesized in the laboratory using standard fmoc chemistry or commercially using the Applied Biosystems 433A peptide synthesizer. Unless otherwise mentioned, chemicals were purchased from Sigma-Aldrich (MilliporeSigma, St. Louis, MO, USA).

As shown in Scheme 5 (below), in a 10 mL solid phase peptide synthesis (SPPS) reactor, 300 mg of Rink amide resin 5a was soaked in DCM for 30 minutes. Rink amide resin 5a was isolated by vacuum filtration. The Rink amide resin 5a was combined with 3 mL 20% piperidine in DMF with a 30-minute exposure time performed in three steps (10 minutes per exposure). After each exposure, the resin was isolated by vacuum filtration and washed three times with methanol and five times with DCM. After deprotection, the resin was combined with 863 mg Fmoc-Lys (ivDde)-OH 5b, 570 mg HATU, and 502 μL DIPEA in 3 mL of DMF and shaken at RT for 150 minutes. The resulting product 5c was isolated by vacuum filtration and washed three times with DMF, six times alternatively with DCM/Methanol, and five times with DCM. Reaction completion was tested using the standard Kaiser test (ninhydrin test). To remove the fmoc protecting group. 5c was combined with 3 mL 20% piperidine in DMF with a 30-minute exposure time performed in three steps (10 minutes per exposure). After deprotection, the resin was washed three times with DMF, six times alternatively with DCM/Methanol, and five times with DCM combined with 523 mg fmoc-L-glutamic acid, alpha-tert-butyl ester 5d. 553 mg HATU, and 502 μL DIPEA in 3 mL of DMF and shaken at RT for 150 minutes. The resulting product 5e was isolated by vacuum filtration and washed three times with DMF, six times alternatively with DCM/Methanol, and five times with DCM. Reaction completion was tested using the standard Kaiser test (ninhydrin test). To remove the fmoc protecting group. 5e was combined with 3 mL 20% piperidine in DMF with a 30-minute exposure time performed in three steps (10 minutes per exposure). After deprotection, the resin was washed three times with DMF, six times alternatively with DCM/Methanol, and five times with DCM and combined with 522 mg IAA and 980 μL DIPEA in 2 mL DMF, and the mixture was shaken at RT overnight. The resulting product 5f was isolated by vacuum filtration and washed three times with DMF, six times alternatively with DCM/Methanol, and five times with DCM. Reaction completion was tested using the standard Kaiser test (ninhydrin test). To remove the fmoc and iv.Dde protecting groups, 4i was combined with 5 mL of 2% hydrazine in DMF with a 9-minute exposure time performed in three steps (3 minutes per exposure). After each exposure, the resin was isolated by vacuum filtration and washed three times with DMF, six times alternatively with DCM/Methanol, and five times with DCM. The resin was combined with a mixture of TFA/H₂O/TIS (95:2.5:2.5) in the peptide shaker and shaken at RT for 3 hours. The resulting product 5g was precipitated in ether.

Afterwards, a solution consisting of 203.6 mg CMD5 5h. 23.8 mg EDC, and 13.8 mg NHS dissolved in 10 mL of deionized water (DI H₂O) was added a solution containing 42.6 mg of TTEA Si dissolved in 10 mL of DMF. The resulting mixture was stirred overnight at RT using a magnetic stirrer. The product was then precipitated in 80 mL acetone and isolated using a centrifuge (2500 rpm, for 10 min), washed with 20 mL acetone, and dried under vacuum for 3 h to obtain a white powder. The white powder was dissolved in a 5 to 10 mL cocktail of TFA/H₂O/TIS (95:2.5:2.5) for 2 hours. Then, 5j was precipitated in diethyl ether, dried under vacuum for 3 h, and stored in a −20 freezer under a nitrogen atmosphere.

In the next step, 5j was dissolved in DI H₂O at a concentration of 1 mM, and applied on a gold surface of the SPR sensors, and incubated overnight at RT. The next day, the sensors were washed with DI H₂O thoroughly to remove any unbonded 5j and dried using nitrogen flow. Then the surface was activated by incubation in a solution of EDC/NHS 0.25 M:0.1M for 1 hour and washed with DI water. The sensors were reacted with Nα, Nα-bis(carboxymethyl)-L-lysine hydrate 5l 100 mM solution consisting of IPA:MQ:Et3N 63:32:5 at RT overnight. CMD-NTA modified sensors 5m were washed with IPA/DI water, dried using nitrogen flow, and stored in dry conditions.

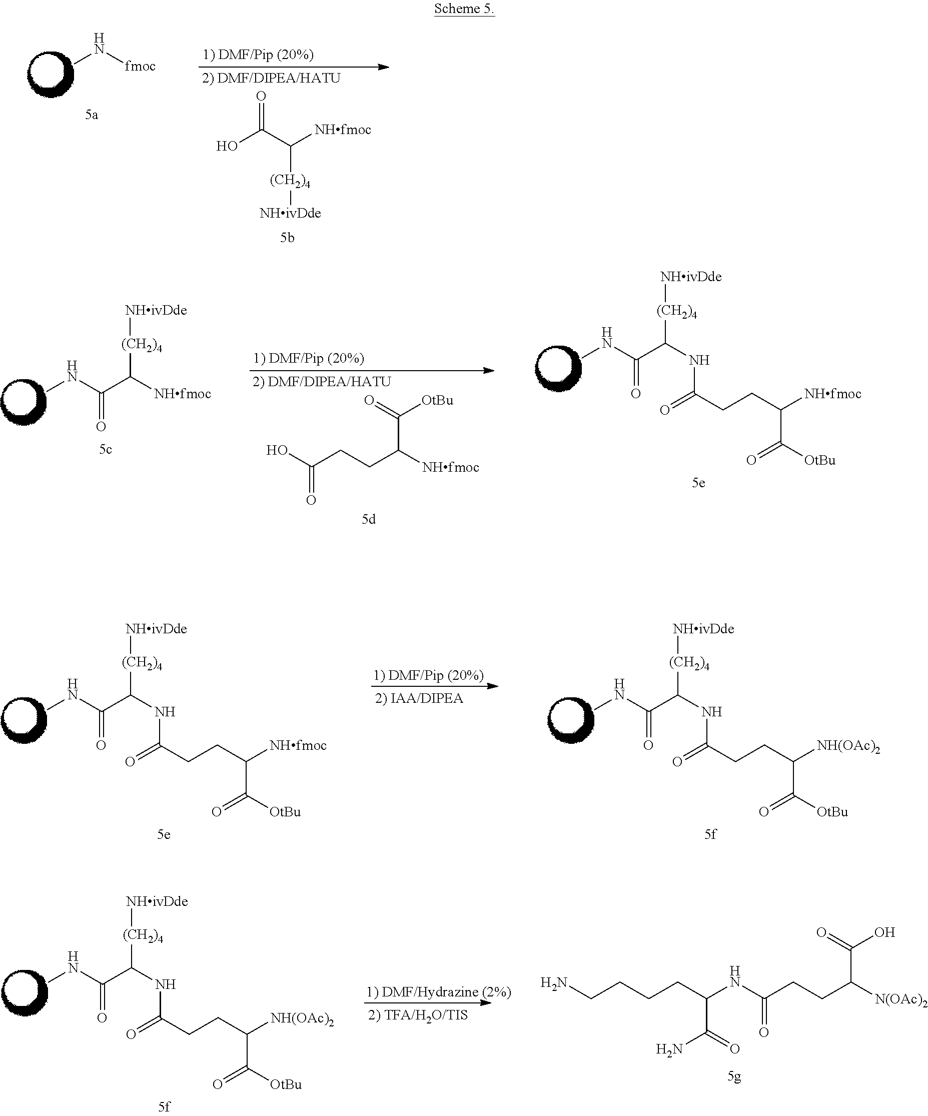

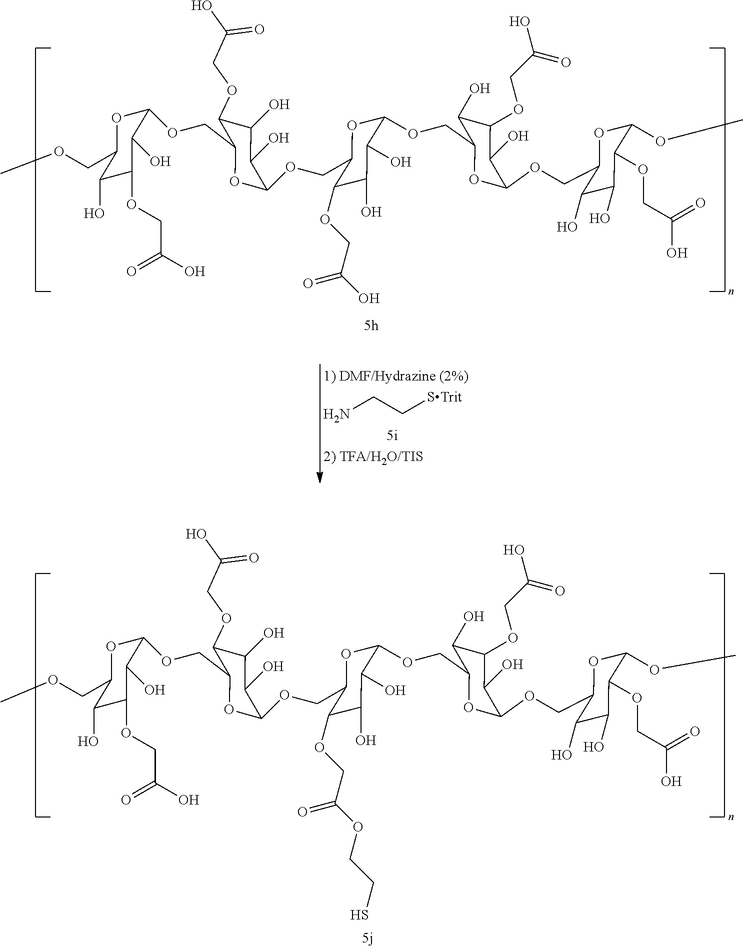

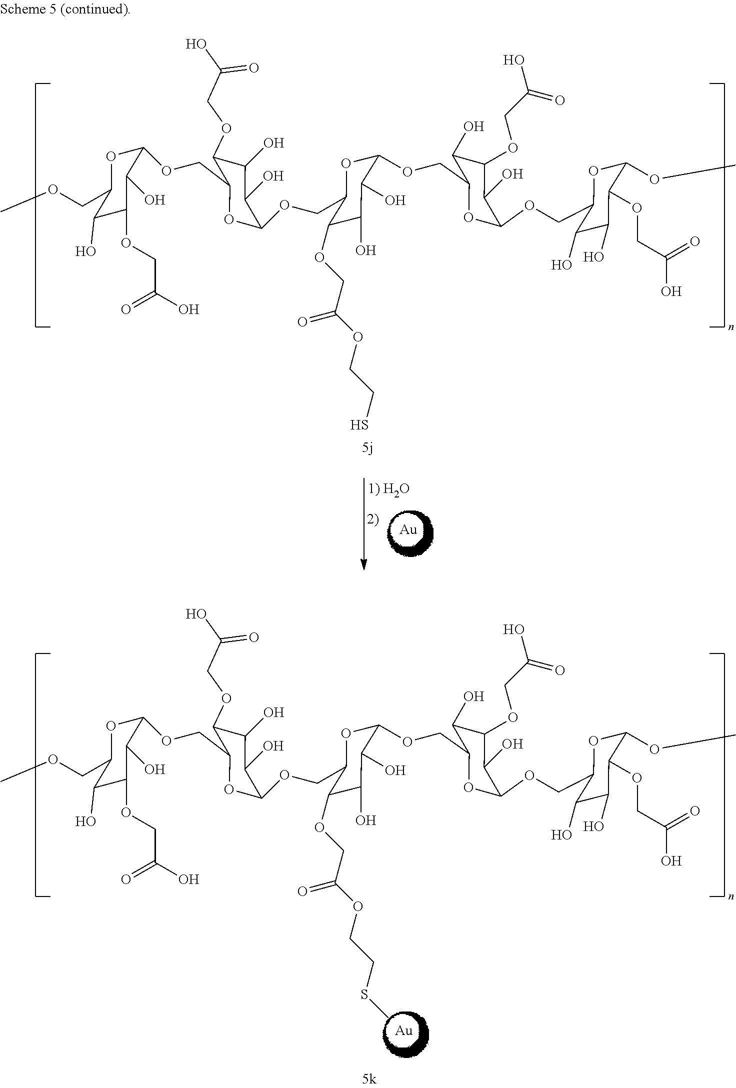

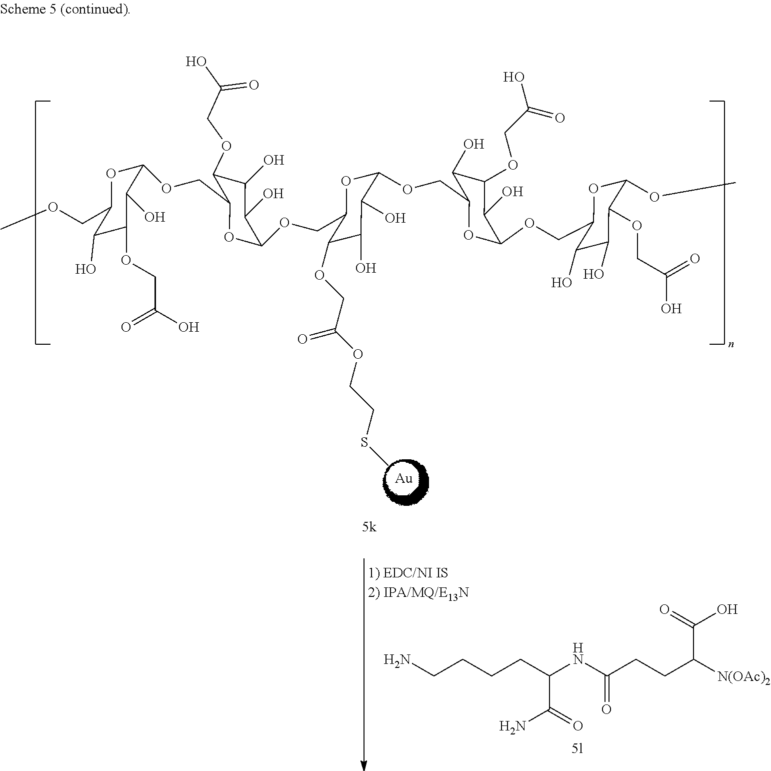

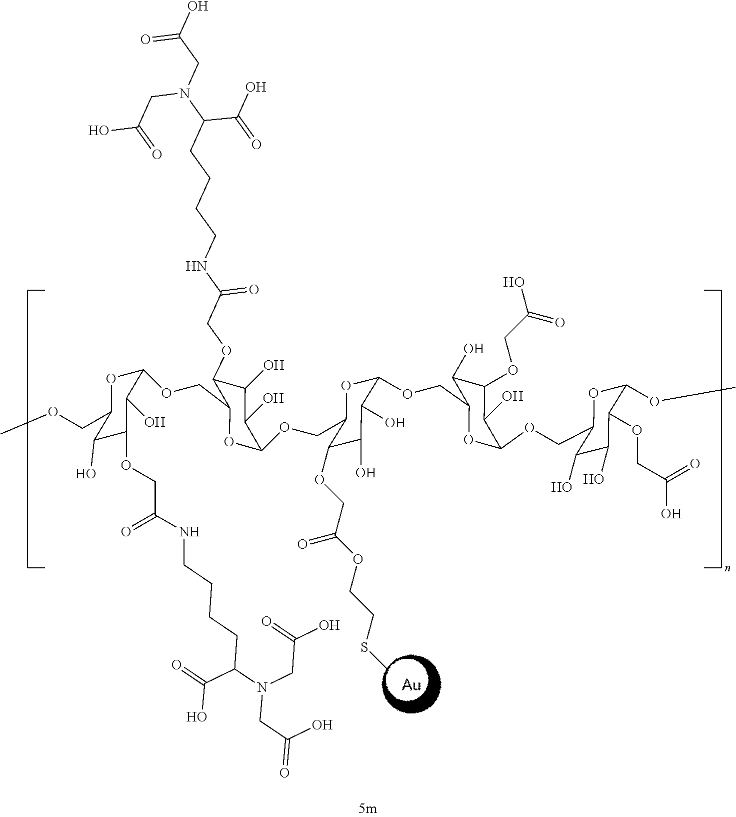

### Example 7—Detection of IL-6/IL-6R Using CMD-NTA

Au NPs coated with CMD-NTA were prepared as described in Example 6 (above). The Au NPs were deposited over sensor chips and integrated into an LSPR system in a reference channel (8 channels) and a sample channel (8 channels).

The sensors were exposed to a series of solutions inside the microfluidic cartridge system. The solution volume of each exposure is around 2 droplet units (“du”) in the microfluidic system which is equal to about 1.4 μL and dispenses from the solution reservoir inside the cartridge.

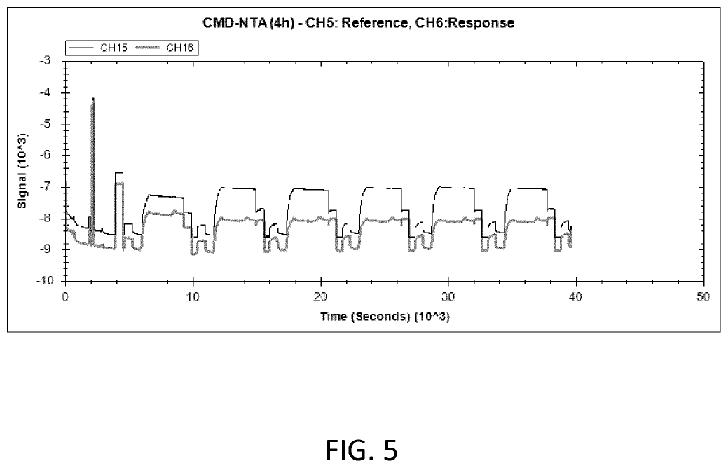

The sensor tips were exposed to PBS buffer at pH of 7.4 containing 0.1% T20 surfactant, as a washing step, which can be observed as a smallpeak around 500 seconds in FIG. 5.

The solutions of 4% and 32% glycerol were introduced to calibrate the system, which can be observed as the first and second peaks, respectively, around 2000 seconds in FIG. 5.

Then the sensors were exposed to a 10 mM HCl solution for cleaning purposes, which can be observed as a peak around 3000 seconds in FIG. 5.

Then the sensors were exposed to a 0.35 M EDTA solution (pH 8) containing 0.1% T20 to remove any possible transition metal from the surface, which can be observed as peaks around 4000 seconds in FIG. 5.

A solution of 40 mM, NiCl2 in water was injected to form the chelate with NTA functionalities, which can be observed as peaks around 5000 seconds in FIG. 5.

Next, a solution of anti-IL-6 ligand (IL-6R Protein Human Recombinant (His-Tag) 40 kDA, PBS pH 7.4) was introduced over the response channels (gray trace) and his-strep protein (0.5 mg/ml) in the reference channels (black trace), which can be observed as peaks at around 6000 seconds in FIG. 5.

Then IL-6 analyte was introduced to both reference and response channels causing the signal to appear as a peak in the response channel (gray trace around 8500 seconds in FIG. 5). This region of the sensorgram is flat in reference channel confirming the positive response over IL-6 in this sensor configuration (black trace around 8500 seconds in FIG. 5).

To show the reversibility of the immobilization and reusability of the senor, the surface was regenerated by exposure to a 10 mM HCL solution (which can be observed as a valley at around 9000 seconds in FIG. 5), and subsequent exposure to a 0.5 M imidazole solution in DI H₂O (which can be observed as peaks at around 1100 seconds in FIG. 5). The optical signal shifted back to the baseline, indicative of an absence of IL-6 on the linker surface.

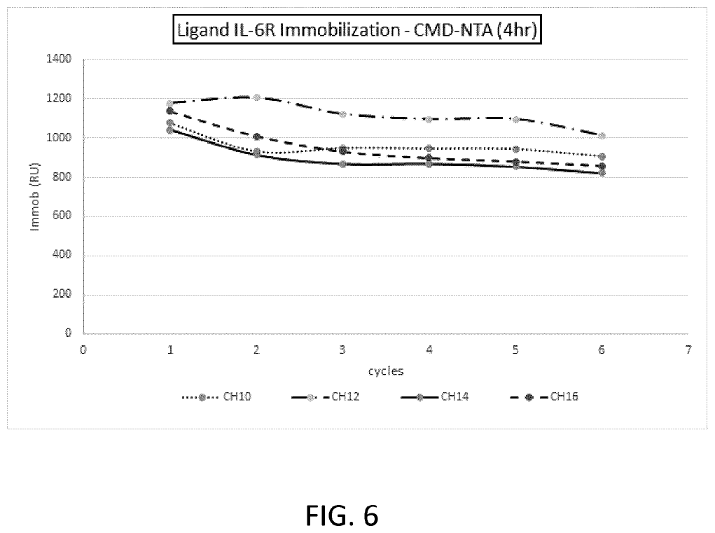

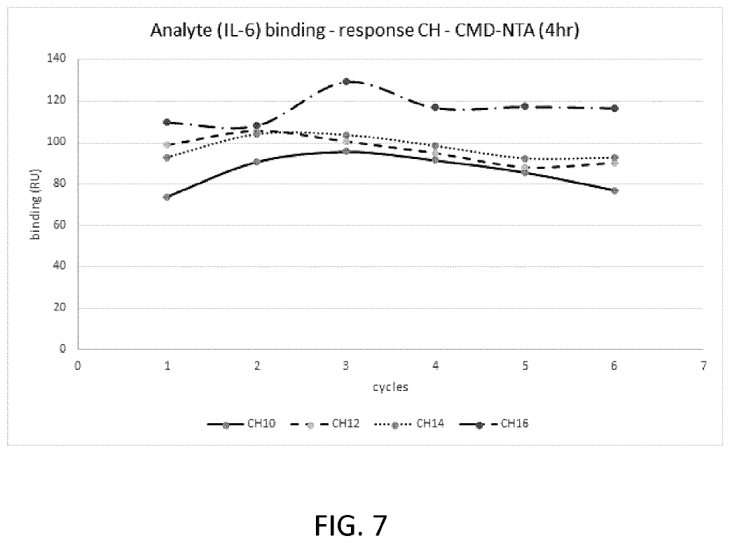

Exposure to the ligand and regeneration were repeated five times. FIG. 6 is a graph showing IL-6 ligand immobilization response for four separate channels. FIG. 7 is a graph showing IL-6 analyte immobilization response for four separate channels.

While preferred embodiments of the present disclosure have been shown and described herein, it will be obvious to those skilled in the art that such embodiments are provided by way of example only. It is not intended that the disclosure be limited by the specific examples provided within the specification. While the disclosure has been described with reference to the aforementioned specification, the descriptions and illustrations of the embodiments herein are not meant to be construed in a limiting sense. Numerous variations, changes, and substitutions will now occur to those skilled in the art without departing from the disclosure. Furthermore, it shall be understood that all aspects of the disclosure are not limited to the specific depictions, configurations or relative proportions set forth herein which depend upon a variety of conditions and variables. It should be understood that various alternatives to the embodiments of the disclosure described herein may be employed in practicing the disclosure. It is therefore contemplated that the invention shall also cover any such alternatives, modifications, variations or equivalents. It is intended that the following claims define the scope of the disclosure and that methods and structures within the scope of these claims and their equivalents be covered thereby.

1. A nitrilotriacetic acid linker having the formula: 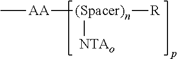
wherein the AA group comprises a linear or branched natural or synthetic amino acid group comprising one or more amino acids;
the optional spacer comprises a linear or branched carbon chain, comprising one or more amino acids, a polymeric moiety, or any combination thereof, wherein in the absence of the spacer, the AA group is coupled to the one or more nitrilotriacetic acid (NTA) groups;
the nitrilotriacetic acid (NTA) group comprises one or more nitrilotriacetic acid functional groups;
wherein R is H, a short chain alkyl, or an NTA group;
wherein n is the number of spacers in the linker;
wherein o is the number of NTA groups in the linker; and
wherein p is the number of 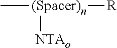
moieties.

2. The nitrilotriacetic acid linker of claim 1, wherein the AA group comprises from one to 10 amino acids.

3. The nitrilotriacetic acid linker of claim 1, wherein the AA group comprises a cysteine or a methionine amino acid.

4. The nitrilotriacetic acid linker of claim 1, wherein the spacer comprises a carbon chain.

5. The nitrilotriacetic acid linker of claim 1, wherein the spacer comprises a PAG group having one or more PAG units.

6. The nitrilotriacetic acid linker of claim 1, wherein the spacer comprises a PEG group having one or more PEG units.

7. The nitrilotriacetic acid linker of claim 1, wherein n comprises from 0 to 50 spacers.

8. The nitrilotriacetic acid linker of claim 1, wherein o comprises from 1 to 10 NTA groups.

9. The nitrilotriacetic acid linker of claim 1, wherein p comprises from 1 to 10 moieties.

10. The nitrilotriacetic acid linker of claim 1, wherein the one or more NTA groups are configured to couple to a ligand and/or an analyte.

11. A surface plasmon resonance (SPR) or localized surface plasmon resonance (LSPR) sensor, the sensor comprising:
(a) a substrate, wherein the substrate comprises a solid support coated with a metal layer; and
(b) a nitrilotriacetic acid (NTA) linker having the formula: 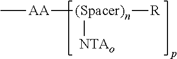
wherein the AA group comprises a linear or branched natural or synthetic amino acid group comprising one or more amino acids;
the optional spacer comprises a linear or branched carbon chain, comprising one or more amino acids, a polymeric moiety, or any combination thereof, wherein in the absence of the spacer, the AA group is coupled to the one or more nitrilotriacetic acid (NTA) groups;
the nitrilotriacetic acid (NTA) group comprises one or more nitrilotriacetic acid functional groups;
wherein R is H, a short chain alkyl, or an NTA group;
wherein n is the number of spacers in the linker;
wherein o is the number of NTA groups in the linker;
wherein p is the number of 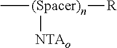
moieties;
(c) wherein the AA group of the NTA linker is coupled to the metal layer; and
(d) wherein the one or more nitrilotriacetic acid functional groups of the linker are capable of binding to one or more ligands and/or analytes.

12. The sensor of claim 11, wherein the sensor comprises a substrate and a coating layer, and the AA group is covalently coupled to the coating layer.

13. The sensor of claim 12, wherein the substrate is selected from a group consisting of silicon substrates, glass substrates, polystyrene substrates, agarose substrates.

14. The sensor of claim 12, wherein the coating layer comprises a first inner coating and a second outer coating.

15. The sensor of claim 14, wherein the first inner coating comprises a polyelectrolyte or a poly(allylamine hydrochloride).

16. The sensor of claim 14, wherein the second outer coating comprises a metal coating, a gold film, a metal nanoparticle coating, or a gold nanoparticle coating.

17. A method for detection of an analyte in a fluid using a surface plasmon resonance (SPR or LSPR) sensor, comprising:
providing an SPR or LSPR sensor comprising a surface, the surface comprising a solid support coated with a metal layer and having a nitrilotriacetic acid linker attached to the metal layer, the nitrilotriacetic acid linker having the formula: 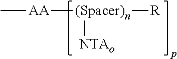
wherein the AA group comprises a linear or branched natural or synthetic amino acid group comprising one or more amino acids;
the optional spacer comprises a linear or branched carbon chain, comprising one or more amino acids, a polymeric moiety, or any combination thereof, wherein in the absence of the spacer, the AA group is coupled to the one or more nitrilotriacetic acid (NTA) groups;
the nitrilotriacetic acid (NTA) group comprises one or more nitrilotriacetic acid functional groups;
wherein R is H, a short chain alkyl, or an NTA group;
wherein n is the number of spacers in the linker;
wherein o is the number of NTA groups in the linker;
wherein p is the number of 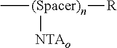
moieties; and
optionally one or more ligands coupled to the nitrilotriacetic acid linker;
contacting a fluid comprising an analyte with the SPR or LSPR sensor; and
measuring an optical signal to detect a change in the optical signal in response to the contacting to measure the analyte in the fluid

18.-66. (canceled)
